(19) United States
(12) Patent Application Publication
(10) Pub. No.: US 2024/0284882 A1
(43) Pub. Date: Aug. 29, 2024

(54) METHOD AND USE OF A TRANSGENIC MOUSE LINE

(71) Applicant: FUJIFILM IRVINE SCIENTIFIC, INC., Santa Ana, CA (US)

(72) Inventors: Kumi Sakurai, Santa Ana, CA (US); Anne Qiu Tran, Santa Ana, CA (US)

(73) Assignee: FUJIFILM IRVINE SCIENTIFIC, INC., Santa Ana, CA (US)

(21) Appl. No.: 18/571,618

(22) PCT Filed: Jun. 21, 2022
(86) PCT No.: PCT/US2022/034294

§ 371 Date: Dec. 18, 2023

Related U.S. Application Data
(60) Provisional application No. 63/213,335, filed on Jun. 22, 2021.

(51) Int. Cl. A01K 67/0275

(52) U.S. Cl. CPC A01K 67/0275; A01K 2217/052; A01K 2227/105; A01K 2267/0387

(57) ABSTRACT

Disclosed herein, in certain embodiments, is a transgenic mouse expressing a fusion protein comprising OCT+ under a transcriptional control. In some embodiments, also disclosed herein include embryos, stem cells, and germline cells obtained from the transgenic mouse. In additional embodiments, disclosed herein include a method of generating the transgenic mouse and a method of assessing a product using an embryo obtained from the transgenic mouse.

This application is a U.S. national stage application under 35 U.S.C. § 371 of International Application No. PCT/US2022/034294, filed Jun. 21, 2022, which claims priority under 35 U.S.C § 119(e) to U.S. Provisional Application No. 63/213,335, filed Jun. 22, 2021, each of which are hereby incorporated by reference in their entireties.

### BACKGROUND

### Sequence Listing

The instant application contains a Sequence Listing which has been submitted electronically in ASCII format and is hereby incorporated by reference in its entirety. Said ASCII copy, created on Jul. 21, 2022, is named 106556-0182_SL.txt and is 39,310 bytes in size.

Genetically manipulated murine models are important for studying gene functions at a whole animal level. Gene knockout mice representing a loss-of-gene function strategy and transgenic mice representing a gain-of-function approach can be utilized to assess molecular and cellular functions of a gene or protein of interest. In general, transgenic mice can be generated by microinjecting the transgenic construct in a fertilized egg (oocyte or zygote). Alternatively, a retrovirus vector comprising the transgene can be introduced into an egg for subsequent generation of a transgenic mouse.

During development, pre-implantation embryos change rapidly, in just a matter of days, from a metabolically quiescent, undifferentiated single cell under the genetic control of maternal transcripts into a dynamic, multi-celled embryo that has developed homeostatic mechanisms and its own functioning genome (Leese 1991; Lane 2001; Gardner et al. 2005). The early embryo, which depends on a pyruvate-based metabolism and is solely dependent on mitochondrial oxidative phosphorylation for energy production; like a unicellular organism, the early embryo lacks many key regulatory functions for pH and osmotic control. After compaction at the eight- to 16-cell stage, there is a change in metabolic control to a highly glycolytic metabolism. Concomitantly, there is also a marked transition in the functional complexity of other cellular mechanisms as the embryo's physiology becomes more like that of a somatic cell. It is the initially crude nature of homeostatic regulation in the early embryo and its subsequent development through later stages of pre-implantation development that pose significant challenges in the laboratory. Maintenance of a favorable in vitro environment, in particular with modulations of one or more genes or proteins of interest, is essential for maximizing viability and promoting ongoing development.

Perturbations to the environment surrounding the embryo during development in culture, relative to “normal” conditions encountered in the reproductive tract, result in reduced embryo viability and impaired development. As such, there is a need for a sensitive and reproducible method and assay for assessing embryo development and toxicity.

### SUMMARY OF THE INVENTION

In certain embodiments, disclosed herein is a transgenic mouse expressing a fusion protein comprising OCT4 under a transcriptional control. In some embodiments, also disclosed herein include embryos, stem cells, and germline cells obtained from the transgenic mouse. In additional embodiments, disclosed herein include a method of generating the transgenic mouse and a method of assessing a product using an embryo obtained from the transgenic mouse.

In some embodiments, disclosed herein is a transgenic mouse comprising stable expression of a fusion protein comprising octamer-binding transcription factor 4 (OCT4) under transcriptional control. In some instances, gene expression of said fusion protein is stably transmitted through germline DNA. In some instances, an embryo expressing an OCT4::EGFP fusion protein can be generated, in which an oocyte is fertilized with a sperm comprising the OCT4::EGFP fusion protein, and the sperm is derived from the transgenic mouse. In some instances, a stem cell expressing an OCT4::EGFP fusion protein is derived from the transgenic mouse. In some cases, a germline cell expressing an OCT4::EGFP fusion protein is derived from the transgenic mouse.

In some embodiments, also disclosed herein is a method of producing a transgenic mouse comprising, microinjection of a zygote with a bacterial artificial chromosome (BAC) construct, wherein the construct comprises a reporter gene operably linked to a mouse OCT4 locus and the zygote is implanted into the reproductive tract of a surrogate mouse, thereby producing the transgenic mouse.

In some embodiments, additionally disclosed herein is a method for assessing a product used for assisted reproductive technologies (ART), treatment of a disease, drug screening, or immune modulation, comprising: (a) obtaining a transgenic embryo comprising stable expression of a fusion protein comprising OCT4; (b) culturing the transgenic embryo; (c) evaluating expression of the fusion protein; and (d) determining acceptability or failure of the product.

In some embodiments, further disclosed herein is a kit comprising the transgenic mouse described herein, the embryo described herein, the stem cell described herein, or the germline cell described herein, optionally comprising an instruction for use.

### BRIEF DESCRIPTION OF THE DRAWINGS

FIG. 1A-FIG. 1B show the effects of suboptimal oil exposure to a transgenic embryo described herein and a control embryo at 48 hours. FIG. 1A illustrates the study protocol. Method A refers to the study protocol using the transgenic embryo described herein. Method B refers to the study protocol using the control embryo. FIG. 1B shows a comparison of detected blastomeres between the transgenic embryo described herein and the control embryo.

FIG. 2A-FIG. 2C show the effect of suboptimal conditions in a cryopreserved transgenic embryo described herein and a control embryo. FIG. 2A illustrates the study design. Method A refers to the study protocol using the transgenic embryo described herein. MEA refers to the mouse embryo assay using the control embryo. A comparison of detected blastomeres between the transgenic embryo described herein and the control embryo is shown at 48 hours (FIG. 2B) and 96 hours (FIG. 2C).

FIG. 3 shows abnormal expression of OCT4-GFP in a transgenic embryo described herein and a control embryo cultured in expired ART medium A at 48 hours. Method A refers to the use of the transgenic embryo described herein. MEA refers to the mouse embryo assay using the control embryo.

FIG. 4 shows abnormal expression of OCT4-GFP in a transgenic embryo described herein and a control embryo cultured in expired ART medium A at 96 hours. Method A refers to the use of the transgenic embryo described herein. MEA refers to the mouse embryo assay using the control embryo.

### DETAILED DESCRIPTION

### Definitions

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the disclosure to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the disclosure. All publications, patent applications, patents and other references mentioned herein are incorporated by reference in their entirety.

The practice of the present technology will employ, unless otherwise indicated, conventional techniques of tissue culture, organic chemistry pharmacology, immunology, molecular biology, microbiology, cell biology, and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the description of the disclosure and the appended claims, the singular forms “a,” “an” and “the” are intended to include the plural forms as well, unless the context clearly indicates otherwise.

As used herein, the term “about” refers to a measurable value such as an amount or concentration and the like, is meant to encompass variations of 20%, 10%, 5%, 1%, 0.5%, or even 0.1% of the specified amount.

As used herein, the term “comprising” is intended to mean that the compositions and methods include the recited elements, but not excluding others. As used herein, the term “consisting essentially of” when used to define compositions and methods, shall mean excluding other elements of any essential significance to the combination. For example, a composition or method consisting essentially of the elements as defined herein would not exclude other elements that do not materially affect the basic and novel characteristic(s) of the claimed invention. As used herein, “consisting of” shall mean excluding more than trace amounts of other ingredients and substantial method steps recited. Embodiments defined by each of these transition terms are within the scope of this disclosure.

As used herein, the terms “acceptable,” “effective,” or “sufficient” refer to the selection of any components, ranges, dose forms, etc. disclosed herein intend that said component, range, dose form, etc. is suitable for the disclosed purpose.

As used herein, “and/or” refers to and encompasses any and all possible combinations of one or more of the associated listed items, as well as the lack of combinations when interpreted in the alternative (“or”).

As used herein, the terms “nucleic acid sequence,” “nucleic acid molecule,” or “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, or alternatively consisting essentially of, or yet further consisting of purine and pyrimidine bases or other natural, chemically or biochemically modified, non-natural, or derivatized nucleotide bases.

As used herein, the term “enhancer” refers to a region of DNA sequence that encodes for a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. As used herein, the term “promoter” refers to a DNA sequence that contains an RNA polymerase binding site, a transcription start site, and/or a TATA box and assists or promotes the transcription and expression of an associated transcribable polynucleotide sequence and/or gene.

As used herein, “under transcriptional control” is a term well understood in the art and indicates that transcription of a polynucleotide sequence, usually a DNA sequence, depends on its being operatively linked to an element which contributes to the initiation of, or promotes, transcription.

As used herein, the term “polypeptide” refers to a chain of at least two covalently linked amino acids. Polypeptides can be encoded by polynucleotides provided herein. Proteins provided herein can be encoded by nucleic acid sequences provided herein. Proteins can comprise polypeptides or amino acid sequences provided herein. As used herein, a “protein” refers to a chain of amino acid residues that are capable of providing structure or enzymatic activity to a cell. As used herein, a “coding sequence” refers to a nucleic acid sequence that encodes a protein.

As used herein, the term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the terms “equivalent” or “biological equivalent” or “similar” are used interchangeably when referring to a particular molecule, biological, or cellular material and intend those having minimal homology while still maintaining desired structure or functionality. Non-limiting examples of equivalent polypeptides, include a polypeptide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide sequences, or a polypeptide which is encoded by a polynucleotide or its complement that hybridizes under conditions of high stringency to a polynucleotide encoding such polypeptide sequences. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity to the reference polynucleotide, e.g., the wild-type polynucleotide.

As used herein, the term “operatively linked” or “operably linked” intends the polynucleotides are arranged in a manner that allows them to function in a cell.

Also as used herein, the term “gene” refers to a polynucleotide containing at least one open reading frame (ORF) that is capable of encoding a particular polypeptide or protein after being transcribed and translated. A “gene product” or alternatively a “gene expression product” refers to the amino acid (e.g., peptide or polypeptide) generated when a gene is transcribed and translated.

As used herein, the term “reporter gene” includes a gene that can be operably linked to the regulatory region of a viability marker and can be visualized or otherwise evaluated to determine its expression. In a preferred embodiment, the reporter gene is a fluorescent or luminescent protein. Fluorescent proteins can include, without limitation, blue/UV proteins such as TagBFP, mTagBFP2, azurite, EBFP2, mKalama1, Sirius, sapphire, and T-sapphire; cyan proteins such as ECFP, cerulean, SCFP3A, mTurquoise, m Turquoise2, monomeric Midoriishi-Cyan, TagCFP, and mTFP1; green proteins such as EGFP, Emerald, Superfolder GFP, Monomeric Azami Green, TagGFP2, mUKG, mWasabi, or Clover; yellow fluorescent proteins such as EYFP, Citrine, Venus, SYFP2, ZsYellow1, and TagYFP; orange proteins for use as reporter genes can include Monomeric Kusabira-Orange, mKOk, mKO2, mOrange, and mOrange2; red proteins such as HcRed1, mRaspberry, mCherry, mStrawberry, mTangerine, tdTomato, TagRFP, mApple, mRuby, and mRuby2; and far-red proteins include, without limitation, mPlum, HcRed-Tandem, mKate2, mNeptune, and NirFP. In some embodiments, the fluorescent protein is selected from green fluorescent protein (GFP), red fluorescent protein (RFP), a yellow fluorescent protein (YPE), or a cyan fluorescent protein (CFP). In some embodiments, the reporter gene may be or include, for example, an epitope tag (e.g. HIS, FLAG, HA) that is recognized by an antibody.

As used herein, the term “linker” refers to an amino acid or peptidomimetic sequence. In some embodiments, linkers have one or more properties that include a flexible conformation, an inability to form an ordered secondary structure or a hydrophobic or charged flexible character which could promote or interact with each domain. Amino acids typically found in flexible protein region include, but not limited to, Gly, Asn, and Ser. The length of the linker sequence may vary without significantly affecting a function or activity.

As used herein, the term “fusion protein” refers to a protein of at least two domains that are encode by separate that have been joined so they are transcribed and translated as a single protein.

As used herein, the term “mutation” refers to an alteration in the nucleotide sequence of the genome of an organism, virus, or extrachromosomal DNA.

As used herein, the term “stably expresses” or “stably express” refers to integration of foreign gene in to the genome.

As used herein, the term “C-terminus,” “carboxyl-terminus,” carboxy-terminus,” “C-terminal tail,” “C-terminal end,” or “COOH-terminus” refers to the end of an amino acid chain terminated by a free carboxyl group (—COOH). As used herein, the term “N-terminus,” “amino-terminus,” “NH₂-terminus,” “N-terminal end,” or “amine-terminus” refers to the start of an amino acid chain referring to the free amine group (—NH₂). When protein is translated from messenger RNA, it is created from N-terminus to the C-terminus.

As used herein, the term “bacterial artificial chromosome construct” or “BAC construct” refers to a DNA construct used for transforming and cloning in bacteria.

As used herein, the term “germline” refers to a population of multicellular organisms cells that pass their genetic material to the progeny. In some embodiment the germline are the cells that form the egg, sperm and the fertilized egg.

As used herein, the term “culturing” refers to the in vitro propagation of cells or organisms on or in media of various kinds. It is understood that the descendants of a cell grown in culture may not be completely identical (i.e., morphologically, genetically, or phenotypically) to the parent cell.

As used herein, the term “mammal” refers to any species classified in the class Mammalia.

As used herein, the term “mouse” refers to a Mus musculus.

As used herein, the term “viable” refers to and animal or cell that can survive or live under a particular environmental condition.

As used herein, the term “fertile” refers to the ability to be able to produce offspring.

As used herein, the term “offspring” or “progeny” refers to the young born of living organisms.

As used herein, the term “reproductive tract” or “reproductive system” refers to a series of organs that contribute to and aid in the reproductive process.

As used herein, the term “surrogate” refers to a female animal that is impregnated by embryo transfer or artificial insemination to bear offspring in place of another animal.

As used herein, the term “transgenic” refers to a segment of DNA that has been incorporated into a host genome or is capable of replication in a host cell and is capable of causing expression of one or more cellular products. Exemplary transgenes can provide the host cell, or animal developed therefrom, with a novel phenotype relative to the corresponding no transformed cell or animal. As used herein, the term “transgenic animal” refers to a non-human animal, usually a mammal, having a non-endogenous nucleic acid sequence present as an extrachromosomal element in at least a portion of its cells or stably integrated into its germ line DNA. In some embodiments, a transgenic animal is a transgenic mouse.

Transgenesis is used to create transgenic mammals such as mice with reporter genes linked to a gene of interest. Methods in molecular genetics and genetic engineering are described generally in the current editions of Molecular Cloning: A Laboratory Manual, (Sambrook et al.); Oligonucleotide Synthesis (M. J. Gait, ed.); Animal Cell Culture (R. I. Freshney, ed.); Gene Transfer Vectors for Mammalian Cells (Miller & Calos, eds.); Current Protocols in Molecular Biology and Short Protocols in Molecular Biology, 3.sup.rd Edition (F. M. Ausubel et al., eds.); and Recombinant DNA Methodology (R. Wu ed., Academic Press). Thus, transgenic technology is well established. See, e.g. Transgenic Mouse: Methods and Protocols (M. Hofker and J. Deursen, Eds.) in Methods in Molecular Biology (Vol. 209) (the contents of which are hereby incorporated by reference in their entirety).

As used herein, the term “microinjection” refers to the use of a glass micropipette to inject a substance at a microscopic level.

As used herein, the term, “Assisted Reproductive Technology” or “ART” as used herein, includes all fertility treatments in which both female gametes (eggs or oocytes) and male gametes (sperm) are handled. In Vitro Fertilization (IVF) is one of several assisted reproductive techniques used to assist infertile couples in conceiving a child. IVF refers to the procedure by which eggs are removed from the female's ovary and fertilized with sperm in a laboratory procedure. The fertilized egg (embryo) can be cryopreserved for future use or transferred to the uterus.

As used herein, “morula” refers to an early-stage embryo comprising about 16 cells in a solid ball contained within the zona pellucida. The morula can also be referred to as a blastomere.

As used herein, “blastocyst” refers to a structure in early embryonic development consisting of a ball of cells with surrounding wall (trophectoderm or TE) which will form the placenta, a fluid filled cavity (blastocoels) which will form the amniotic sac, and an internal cluster of cells called the inner cell mass (ICM) from which the fetus arises.

As used herein, octamer-binding transcription factor 4 (Oct-4 or OCT4; also referred to as POU domain, class 5, transcription factor 1 (POU5F1)) is a protein that is involved in the self-renewal of undifferentiated embryonic stem cells. OCT4 contains three domains, a N-terminal domain, a POU domain, and a C-terminal domain. Both the N-terminal and C-terminal domains are involved in transactivation, but the activity of the C-terminal domain is cell type specific and is regulated through phosphorylation. The POU-domain functions as an interaction site for binding by cell type-specific regulatory factors.

Mouse embryo assay (MEA) is a functional and toxicological bioassay utilised to detect toxicity and suboptimal compounds. The MEA has been the gold standard to examine the applicability of culture media and environment without involving human materials. The basic techniques and protocols employed for performing the MEA are set forth in In Vitro Fertilization and Embryo Transfer: A Manual of Basic Techniques (Don P. Wolf, Editor), 1988, pages 57-75; and Mouse Embryo Assay for Assisted Reproduction Technology Devices: Guidance for Industry and Food and Drug Administration Staff, issued by the U.S. Food and Drug Administration, the contents of which are hereby incorporated by reference in their entirety. Briefly, the assay involves superovulation of female mice with pregnant mare serum gonadotropin (PMSG) and human chorionic gonadotropin (hCG). The mice are placed with males at the time of hCG injection and killed 24 hours following hCG to obtain one-cell embryos or 36 hours after injection to obtain two-cell embryos. One-cell embryos are selected for use if they have two polar bodies visible; two cell embryos are selected for use if they look morphologically normal. To examine whether a test article may present any toxicity to the mouse embryos, the embryos can be incubated in the test article under normal culture conditions (e.g., 37° C. and 5% CO₂) for about 96 hours if a one-cell system is used or 72 hours for a two-cell system. Alternatively, the culture can also be extended to five days, six days, or more. Upon completion of the embryo culture, the embryos can be evaluated for development (e.g., blastocyst development). Acceptance can include 80% or more embryos developed to expanded blastocysts.

### Transgenic Mouse

In certain embodiments, disclosed herein is a transgenic mouse which comprises, consists essentially of, or consists of a stable expression of a fusion protein comprising octamer-binding transcription factor 4 (OCT4). In some instances, the fusion protein is under a transcriptional control. In some instances, the gene expression of the fusion protein is stably transmitted through germline DNA.

In some embodiments, the OCT4 protein is a mouse OCT4. The OCT4 protein can comprise a full-length OCT4, or a fragment thereof, e.g., a functional fragment thereof. As used herein, the term “functional fragment” refers to an OCT4 fragment that is capable of inducing an equivalent function as the wild-type OCT4, for example an equivalent function such as transactivation, self-renewal of undifferentiated embryonic stem cells, and/or pluripotency of the embryonic cells. In some instances, the OCT4 protein comprises a deletion (e.g., of 1, 2, 3, 4, 5, 6, 7, 8, 9, 10, 15, 20, 25, 30, 40, 50, or more residues) at the N-terminus, the C-terminus, and/or an internal region within the protein. In some instances, the OCT4 protein comprises a deletion of a domain, e.g., a deletion of the N-terminal domain, the C-terminal domain, and/or the POU domain. In some cases, the OCT4 protein comprises a wild-type OCT4 protein. In other cases, the OCT4 protein comprises one or more mutations, e.g., 1, 2, 3, 4, 5, 6, 7, 8, 9, 10, or more mutations.

The OCT4 protein can comprise at least or about 70% sequence identity or similarity to SEQ ID NO: 1. In some cases, the OCT4 protein comprises at least or about 80%, 85%, 90%, 91%, 92%, 93%, 94%, 95%, 96%, 97%, 98%, 99%, or 100% sequence identity or similarity to SEQ ID NO: 1. In some cases, the OCT4 protein comprises at least or about 80% sequence identity to SEQ ID NO: 1. In some cases, the OCT4 protein comprises at least or about 90% sequence identity to SEQ ID NO: 1. In some cases, the OCT4 protein comprises at least or about 95% sequence identity to SEQ ID NO: 1. In some cases, the OCT4 protein comprises at least or about 96% sequence identity to SEQ ID NO: 1. In some cases, the OCT4 protein comprises at least or about 97% sequence identity to SEQ ID NO: 1. In some cases, the OCT4 protein comprises at least or about 98% sequence identity to SEQ ID NO: 1. In some cases, the OCT4 protein comprises at least or about 99% sequence identity to SEQ ID NO: 1. In some cases, the OCT4 protein comprises a sequence as set forth in SEQ ID NO: 1. In some cases, the OCT4 protein consist of SEQ ID NO: 1.

In some embodiments, the fusion protein is a fluorescent tagged OCT4 protein. In some instances, the fluorescent tag is a fluorescent protein comprising a green fluorescent protein (GFP), a red fluorescent protein (RFP), a yellow fluorescent protein (YFP), or a cyan fluorescent protein (CFP). In some cases, the fluorescent protein is a GFP or enhanced green fluorescent protein (eGFP). In some cases, the fluorescent protein is a wild-type protein, e.g., a wild-type GFP or eGFP. In other cases, the fluorescent protein comprises one or more mutations, e.g., one or more mutations within the GFP or eGFP.

In some embodiments, the fluorescent protein is a GFP (e.g., eGFP). In some instances, the GFP (e.g., eGFP) is a full-length GFP. In other instances, the GFP (e.g., eGFP) is a fragment thereof, e.g., a functional fragment thereof. As used herein, the term “functional fragment” refers to a GFP fragment that is capable of producing a fluorescence. In some cases, the GFP (e.g., eGFP) comprises a deletion (e.g., of 1, 2, 3, 4, 5, 6, 7, 8, 9, 10, 15, 20, 25, 30, 40, 50, or more residues) at the N-terminus, the C-terminus, and/or an internal region within the protein. In some cases, the GFP (e.g., eGFP) comprises 1, 2, 3, 4, 5, 6, 7, 8, 9, 10, or more mutations. In some cases, the GFP (e.g., eGFP) comprises an A206K mutation.

In some instances, the fluorescent protein is a GFP comprising at least or about 70% sequence identity or similarity to SEQ ID NO: 2. In some cases, the GFP comprises at least or about 80%, 85%, 90%, 91%, 92%, 93%, 94%, 95%, 96%, 97%, 98%, 99%, or 100% sequence identity or similarity to SEQ ID NO: 2. In some cases, the GFP comprises at least or about 80% sequence identity to SEQ ID NO: 2. In some cases, the GFP comprises at least or about 90% sequence identity to SEQ ID NO: 2. In some cases, the GFP comprises at least or about 95% sequence identity to SEQ ID NO: 2. In some cases, the GFP comprises at least or about 96% sequence identity to SEQ ID NO: 2. In some cases, the GFP comprises at least or about 97% sequence identity to SEQ ID NO: 2. In some cases, the GFP comprises at least or about 98% sequence identity to SEQ ID NO: 2. In some cases, the GFP comprises at least or about 99% sequence identity to SEQ ID NO: 2. In some cases, the GFP comprises a sequence as set forth in SEQ ID NO: 2. In some cases, the GFP consist of SEQ ID NO: 2.

The fluorescent protein (e.g., the GFP or eGFP) can be operably linked to the N-terminus, the C-terminus, or at an internal site of the OCT4 protein. In some cases, the fluorescent protein (e.g., the GFP or eGFP) is operably linked to the C-terminus of the OCT4 protein.

In some embodiments, the germline is selected from, but not limited to, a sperm, oocyte, a stem cell, or zygote. In some cases, the germline is selected from a sperm. In some cases, the germline is selected from an oocyte. In some cases, the germline is selected from a stem cell. In some cases, the germline is selected from a zygote.

In some instances, the transgenic mouse is a viable and fertile mouse. In some instances, the transgenic mouse is a viable male, capable of generating an offspring that comprises the fusion protein that is stably integrated into the offspring. In other instances, the transgenic mouse is a viable female, capable of generating an offspring that comprises the fusion protein that is stably integrated into the offspring.

In some cases, the gene expression of the fusion protein in the zygote starts from a 2-cell stage, 3-cell stage, or 4-cell stage cell development.

In certain embodiments, disclosed herein is a method of producing a transgenic mouse described above. In some embodiments, the method comprises, or alternatively consists essentially of, or yet further consists of, microinjection of a zygote with a construct comprising, or alternatively consisting essentially of, or yet further consisting of a reporter gene operably linked to a mouse OCT4 locus and the zygote is implanted into the reproductive tract of a surrogate mouse, thereby producing the transgenic mouse. In some instances, the construct is a bacterial artificial chromosome (BAC) construct, and the construct comprises or alternatively consisting essentially of, or yet further consisting of a reporter gene operably linked to a mouse OCT4 locus. In some cases, the transgenic mice stably expresses the reporter gene.

In some embodiments, the reporter gene locus is stably transmitted through germline DNA of the transgenic mouse. The germline can be selected from sperm, oocytes, stem cells, or zygotes.

In some embodiments, the reporter gene encodes a fluorescent protein. In some instances, the fluorescent protein is selected from, but not limited to, green fluorescent protein (GFP), a red fluorescent protein (RFP), a yellow fluorescent protein (YFP), or a cyan fluorescent protein (CFP). In one aspect, the GFP is an enhanced green fluorescent protein (eGFP). In one aspect, the eGFP comprises, or alternatively consists essentially of, or yet further consists of an A206K mutation.

In some embodiments, the reporter gene comprise a nucleic acid sequence encoding a fluorescent protein comprising at least or about 70%, 80%, 85%, 90%, 91%, 92%, 93%, 94%, 95%, 96%, 97%, 98%, 99%, or 100% sequence identity or similarity to SEQ ID NO: 2. In some cases, the nucleic acid sequence encodes a fluorescent protein comprising at least or about 80% sequence identity to SEQ ID NO: 2. In some cases, the nucleic acid sequence encodes a fluorescent protein comprising at least or about 85% sequence identity to SEQ ID NO: 2. In some cases, the nucleic acid sequence encodes a fluorescent protein comprising at least or about 90% sequence identity to SEQ ID NO: 2. In some cases, the nucleic acid sequence encodes a fluorescent protein comprising at least or about 95% sequence identity to SEQ ID NO: 2. In some cases, the nucleic acid sequence encodes a fluorescent protein comprising at least or about 96% sequence identity to SEQ ID NO: 2. In some cases, the nucleic acid sequence encodes a fluorescent protein comprising at least or about 97% sequence identity to SEQ ID NO: 2. In some cases, the nucleic acid sequence encodes a fluorescent protein comprising at least or about 98% sequence identity to SEQ ID NO: 2. In some cases, the nucleic acid sequence encodes a fluorescent protein comprising at least or about 99% sequence identity to SEQ ID NO: 2. In some cases, the nucleic acid sequence encodes a fluorescent protein comprising SEQ ID NO: 2. In some cases, the nucleic acid sequence encodes a fluorescent protein consisting of SEQ ID NO: 2.

In some embodiments, the reporter gene is operably linked to a coding sequence. In an aspect, the coding sequence encodes the OCT4 protein. In some cases, the OCT4 protein comprises at least or about 70%, 80%, 85%, 90%, 91%, 92%, 93%, 94%, 95%, 96%, 97%, 98%, 99%, or 100% sequence identity or similarity to SEQ ID NO: 1. In some cases, the OCT4 protein comprises at least or about 80% sequence identity to SEQ ID NO: 1. In some cases, the OCT4 protein comprises at least or about 90% sequence identity to SEQ ID NO: 1. In some cases, the OCT4 protein comprises at least or about 95% sequence identity to SEQ ID NO: 1. In some cases, the OCT4 protein comprises at least or about 96% sequence identity to SEQ ID NO: 1. In some cases, the OCT4 protein comprises at least or about 97% sequence identity to SEQ ID NO: 1. In some cases, the OCT4 protein comprises at least or about 98% sequence identity to SEQ ID NO: 1. In some cases, the OCT4 protein comprises at least or about 99% sequence identity to SEQ ID NO: 1. In some cases, the OCT4 protein comprises a sequence as set forth in SEQ ID NO: 1. In some cases, the OCT4 protein consist of SEQ ID NO: 1.

In some embodiments, the reporter gene and the gene coding sequence (e.g., OT4) are separated by a linker. In one aspect, the linker encodes an amino acid sequence comprising a plurality of Ala, Gly, or a combination thereof. In one aspect, the linker encodes an amino acid sequence comprising a (Gly₄Ser)n linker, in which n is an integer selected from 1-10 (SEQ ID NO: 16); optionally selected from 1-6, 1-4, and 1-3; and further optionally selected from 1, 2, 3, 4, 5, 6, 7, 8, 9, and 10. In one aspect, the linker encodes an amino acid sequence comprising SGGGGSGGGGSGGGGS (SEQ ID NO: 3). In some embodiments, the reporter gene is operably linked to the N-terminus, the C-terminus, or at an internal region of the coding sequence (e.g., OCT4). In an aspect, the linker connects the reporter gene to the C-terminus of the coding sequence (e.g., OCT4).

In some embodiments, the polypeptide comprising the fluorescent protein and the OCT4 protein comprises at least or about 70% sequence identity or similarity to SEQ ID NO: 4. In some instances, the polypeptide comprising the fluorescent protein and the OCT4 protein comprises at least or about 80%, 85%, 90%, 91%, 92%, 93%, 94%, 95%, 96%, 97%, 98%, 99%, or 100% sequence identity or similarity to SEQ ID NO: 4. In some cases, the polypeptide comprising the fluorescent protein and the OCT4 protein comprises at least or about 80% sequence identity to SEQ ID NO: 4. In some cases, the polypeptide comprising the fluorescent protein and the OCT4 protein comprises at least or about 90% sequence identity to SEQ ID NO: 4. In some cases, the polypeptide comprising the fluorescent protein and the OCT4 protein comprises at least or about 95% sequence identity to SEQ ID NO: 4. In some cases, the polypeptide comprising the fluorescent protein and the OCT4 protein comprises at least or about 96% sequence identity to SEQ ID NO: 4. In some cases, the polypeptide comprising the fluorescent protein and the OCT4 protein comprises at least or about 97% sequence identity to SEQ ID NO: 4. In some cases, the polypeptide comprising the fluorescent protein and the OCT4 protein comprises at least or about 98% sequence identity to SEQ ID NO: 4. In some cases, the polypeptide comprising the fluorescent protein and the OCT4 protein comprises at least or about 99% sequence identity to SEQ ID NO: 4. In some cases, the polypeptide comprising the fluorescent protein and the OCT4 protein comprises a sequence as set forth in SEQ ID NO: 4. In some cases, the polypeptide comprising the fluorescent protein and the OCT4 protein consists of SEQ ID NO: 4.

In some embodiments, the construct encodes a OCT4::EGFP fusion protein. In some instances, the construct comprises a nucleic acid sequence comprising at least or about 70% sequence identity or similarity to SEQ ID NO: 5. In some instances, the nucleic acid sequence comprises at least or about 80%, 85%, 90%, 91%, 92%, 93%, 94%, 95%, 96%, 97%, 98%, 99%, or 100% sequence identity or similarity to SEQ ID NO: 5. In some cases, the nucleic acid sequence comprises at least or about 80% sequence identity to SEQ ID NO: 5. In some cases, the nucleic acid sequence comprises at least or about 85% sequence identity to SEQ ID NO: 5. In some cases, the nucleic acid sequence comprises at least or about 90% sequence identity to SEQ ID NO: 5. In some cases, the nucleic acid sequence comprises at least or about 95% sequence identity to SEQ ID NO: 5. In some cases, the nucleic acid sequence comprises at least or about 96% sequence identity to SEQ ID NO: 5. In some cases, the nucleic acid sequence comprises at least or about 97% sequence identity to SEQ ID NO: 5. In some cases, the nucleic acid sequence comprises at least or about 98% sequence identity to SEQ ID NO: 5. In some cases, the nucleic acid sequence comprises at least or about 99% sequence identity to SEQ ID NO: 5. In some cases, the nucleic acid sequence comprises a sequence as set forth in SEQ ID NO: 5. In some cases, the nucleic acid sequence consist of SEQ ID NO: 5.

In some instances, the construct mediates expression of the OCT4::EGFP fusion protein. In some cases, the OCT4::EGFP fusion protein is stably integrated into the zygote.

In some instances, the OCT4 locus within the construct comprises a deletion of a proximal enhancer element.

In some embodiments, disclosed herein is an embryo expressing an OCT4::EGFP fusion protein, in which an oocyte is fertilized with a sperm comprising the OCT4::EGFP fusion protein, and in which the sperm is derived from the transgenic mouse described above.

In some embodiments, disclosed herein is a stem cell expressing an OCT4::EGFP fusion protein derived from the transgenic mouse described above.

In some embodiments, disclosed herein is a germline cell expressing an OCT4::EGFP fusion protein derived from the transgenic mouse described above.

### Methods of Product Assessment

In certain embodiments, disclosed herein is a method for assessing a product used for assisted reproductive technologies (ART), treatment of a disease, drug screening, or immune modulation. In some instances, the method comprises (a) obtaining a transgenic embryo comprising stable expression of a fusion protein comprising OCT4; (b) culturing the transgenic embryo; (c) evaluating expression of the fusion protein; and (d) determining acceptability or failure of the product.

In some embodiments, the fusion protein is a fluorescent protein fused to the OCT4 protein. In some instances, the fluorescent protein is selected from a green fluorescent protein (GFP), a red fluorescent protein (RFP), a yellow fluorescent protein (YFP), or a cyan fluorescent protein (CFP). In some cases, the fluorescent protein is selected from GFP or enhanced green fluorescent protein (eGFP). In some cases, the eGFP comprises a mutation, e.g., an A206K mutation.

In some embodiments, the evaluating step comprises determining a temporal and/or spatial expression pattern of the fusion protein. The evaluating step can comprise visualizing nuclear localization and/or cytoplasm localization of the fusion protein. The nuclear localization can encompass shuttling of the fusion protein into the nucleus, as well as binding of DNA by the fusion protein in the nucleus. The evaluating step can further include comparing the temporal and/or spatial expression pattern of the fusion protein with a control, to determine whether an abnormality has occurred with the embryo development. A control as used herein refers to a temporal and/or spatial expression pattern of the fusion protein from an equivalent embryo in which the embryo has proceed through normal development.

In some cases, the evaluating step occurs at a 4-cell or 8-cell stage. In some cases, the fusion protein is predominately localized in the nucleus at a 4-cell stage. As used herein, the term “predominately” refers to at least or about 50%, 60%, 70%, 80%, 90%, 95%, or more of the fusion protein localized in the nucleus. In some cases, at least or about 50% of the fusion protein is localized in the nucleus. In some cases, at least or about 60% of the fusion protein is localized in the nucleus. In some cases, at least or about 70% of the fusion protein is localized in the nucleus. In some cases, at least or about 80% of the fusion protein is localized in the nucleus. In some cases, at least or about 90% of the fusion protein is localized in the nucleus. In some cases, at least or about 95% of the fusion protein is localized in the nucleus.

In some instances, the evaluating step comprises determining the location of the expression of the fusion protein at a 4-cell or 8-cell stage. In some instances, the fusion protein is predominately expressed in the nucleus at a 4-cell stage (e.g., at least or about 50%, 60%, 70%, 80%, 90%, 95%, or more of the fusion protein expressed in the nucleus). In some cases, at least or about 50% of the fusion protein is expressed in the nucleus. In some cases, at least or about 60% of the fusion protein is expressed in the nucleus. In some cases, at least or about 70% of the fusion protein is expressed in the nucleus. In some cases, at least or about 80% of the fusion protein is expressed in the nucleus. In some cases, at least or about 90% of the fusion protein is expressed in the nucleus. In some cases, at least or about 95% of the fusion protein is expressed in the nucleus.

In some instances, the evaluating step occurs at the 8-cell stage. In some cases, at least or about 80%, 90%, 95%, 99%, or more of the fusion protein is localized in the nucleus. In some cases, at least or about 80% of the fusion protein is localized in the nucleus. In some cases, at least or about 90% of the fusion protein is localized in the nucleus. In some cases, at least or about 95% of the fusion protein is localized in the nucleus. In some cases, about 100% of the fusion protein is localized in the nucleus.

In some instances, the evaluating step comprises determining the location of the expression of the fusion protein at the 8-cell stage. In some cases, at least or about 80%, 90%, 95%, 99%, or more of the fusion protein is expressed in the nucleus. In some cases, at least or about 80% of the fusion protein is expressed in the nucleus. In some cases, at least or about 90% of the fusion protein is expressed in the nucleus. In some cases, at least or about 95% of the fusion protein is expressed in the nucleus. In some cases, about 100% of the fusion protein is expressed in the nucleus.

In some instances, the evaluating step occurs at the morula stage. In some cases, at least or about 80%, 90%, 95%, or more of the fusion protein is localized in the nucleus. In some cases, at least or about 80% or more of the fusion protein is localized in the nucleus. In some cases, at least or about 90% or more of the fusion protein is localized in the nucleus. In some cases, at least or about 95% or more of the fusion protein is localized in the nucleus. In some cases, about 100% of the fusion protein is localized in the nucleus.

In some instances, the evaluating step comprises determining the location of the expression of the fusion protein at the morula stage. In some cases, at least or about 80%, 90%, 95%, or more of the fusion protein is expressed in the nucleus. In some cases, at least or about 80% or more of the fusion protein is expressed in the nucleus. In some cases, at least or about 90% or more of the fusion protein is expressed in the nucleus. In some cases, at least or about 95% or more of the fusion protein is expressed in the nucleus. In some cases, about 100% of the fusion protein is expressed in the nucleus.

In some instances, the evaluating step occurs at the blastocyst stage. In some cases, at least or about 60%, 70%, 80%, 90%, 95%, or more of the fusion protein is localized in the inner cell mass (ICM). In some cases, at least or about 70% or more of the fusion protein is localized in the ICM. In some cases, at least or about 80% or more of the fusion protein is localized in the ICM. In some cases, at least or about 90% or more of the fusion protein is localized in the ICM. In some cases, at least or about 95% or more of the fusion protein is localized in the ICM. In some cases, about 100% of the fusion protein is localized in the ICM. In some cases, the fusion protein is not localized in the trophoblast.

In some instances, the evaluating step comprises determining the location of the expression of the fusion protein at the blastocyst stage. In some cases, at least or about 60%, 70%, 80%, 90%, 95%, or more of the fusion protein is expressed in the inner cell mass (ICM). In some cases, at least or about 70% or more of the fusion protein is expressed in the ICM. In some cases, at least or about 80% or more of the fusion protein is expressed in the ICM. In some cases, at least or about 90% or more of the fusion protein is expressed in the ICM. In some cases, at least or about 95% or more of the fusion protein is expressed in the ICM. In some cases, about 100% of the fusion protein is expressed in the ICM. In some cases, the fusion protein is not expressed in the trophoblast.

In some embodiments, the fusion protein is detectable around from about 24 hours to about 96 hours, from about 24 hours to about 72 hours, from about 24 hours to about 48 hours, from about 24 hours to about 36 hours, from about 36 hours to about 96 hours, from about 36 hours to about 72 hours, from about 36 hours to about 48 hours, from about 48 hours to about 72 hours, or from about 48 hours to about 96 hours of culture. In some cases, the fusion protein is detectable from about 36 hours to about 96 hours of culture. In some cases, the fusion protein is detectable from about 36 hours to about 72 hours of culture. In some cases, the fusion protein is detectable from about 36 hours to about 48 hours of culture. In some cases, the fusion protein is detectable from about 48 hours to about 96 hours of culture. In some cases, the fusion protein is detectable from about 48 hours to about 72 hours of culture. In some instances, the fusion protein is detected through visual inspection, e.g., detected based on the fluorescence of the fluorescent protein. In other instances, the fusion protein is detected through nucleic acid expression analysis. In additional instances, the fusion protein is detected through protein expression analysis.

In some instances, the fusion protein is detectable at about 24 hours, about 36 hours, about 48 hours, about 72 hours, or about 96 hours of culture. In some cases, the fusion protein is detectable at about 36 hours of culture. In some cases, the fusion protein is detectable at about 48 hours of culture. In some cases, the fusion protein is detectable at about 72 hours of culture. In some cases, the fusion protein is detectable at about 96 hours of culture. In some instances, the fusion protein is detected through visual inspection, e.g., detected based on the fluorescence of the fluorescent protein. In other instances, the fusion protein is detected through nucleic acid expression analysis. In additional instances, the fusion protein is detected through protein expression analysis.

In some embodiments, the fusion protein is detectable at the 2-cell stage, 3-cell stage, 4-cell stage, 8-cell stage, 16-cell stage, morula stage, or the blastocyst. In some embodiments, the fusion protein is detectable at the 2-cell stage, 3-cell stage, 4-cell stage, or 8-cell stage cell development. In some cases, the fusion protein is detectable at the 4-cell stage cell development. In some cases, the fusion protein is detectable at the 8-cell stage cell development. In some cases, the fusion protein is detectable at the 16-cell stage cell development. In some cases, the fusion protein is detectable at the morula stage cell development. In some cases, the fusion protein is detectable at the blastocyst stage cell development. In some instances, the fusion protein is detected through visual inspection, e.g., detected based on the fluorescence of the fluorescent protein. In other instances, the fusion protein is detected through nucleic acid expression analysis. In additional instances, the fusion protein is detected through protein expression analysis.

In some instances, the evaluating step occurs once a day, twice a day, three times a day, every other day, or on each consecutive days during the culturing process. In some cases, one or more evaluating steps occur from about 24 hours to about 96 hours, from about 24 hours to about 72 hours, from about 24 hours to about 48 hours, from about 24 hours to about 36 hours, from about 36 hours to about 96 hours, from about 36 hours to about 72 hours, from about 36 hours to about 48 hours, from about 48 hours to about 72 hours, or from about 48 hours to about 96 hours from the start of the culturing process.

In some embodiments, the evaluating step can include, for example, one or more of: a) capturing at least one image of the transgenic embryo at a particular developmental stage, b) determining the location of the fusion protein based on the image; and c) comparing the location of the fusion protein to a control. The control can be the location of the fusion protein in an equivalent transgenic embryo at the particular developmental stage and the equivalent transgenic embryo has proceeded to a normal embryo development.

In some embodiments, the evaluating step further comprises determining the expression level of the fusion protein with the control. In some cases, the expression level is determined by measuring the light emission and/or intensity visually, or using a device for the same, by determining the nucleic acid expression, or by determining the protein expression.

In some instances, the product is acceptable if there is nuclear localization or expression of the fusion protein, e.g., at the 4-cell stage, 8-cell stage, or the morula stage. In some instances, the product is acceptable if there is localization or expression in the ICM during the blastocyst stage.

In some cases, the product is not acceptable if there is less than 40%, 30%, 20%, 10%, 5%, or 1% of nuclear localization or expression of the fusion protein at the 4-cell or 8-cell stage. In some cases, the product is not acceptable if there is no nuclear localization or expression of the fusion protein at the 8-cell stage.

In some cases, the product is not acceptable if there is less than 40%, 30%, 20%, 10%, 5%, or 1% of nuclear localization or expression of the fusion protein at the morula stage. In some cases, the product is not acceptable if there is no nuclear localization or expression of the fusion protein at the morula stage.

In some cases, the product is not acceptable if there is less than 40%, 30%, 20%, 10%, 5%, or 1% of localization or expression of the fusion protein in the ICM at the blastocyst stage. In some cases, the product is not acceptable if there is no localization or expression of the fusion protein in the ICM at the blastocyst stage. In some cases, the product is not acceptable if there is localization or expression of the fusion protein in the trophoblast at the blastocyst stage.

In some embodiments, the product is for use with assisted reproductive technologies (ART). The product can include consumables, that include, without limitation, media, media supplements, plastic ware, tubing, pipettes, pipette tips, etc. or any material that comes into contact with the eggs or embryos. Plastic and glassware can include assisted reproduction needles, laboratory gloves, assisted reproduction catheters, and assisted reproduction microtools such as pipettes or other devices used in the laboratory to denude, micromanipulate, hold, or transfer embryos. IVF consumables further include assisted reproduction labware, including without limitation, syringes, IVF tissue culture dishes, IVF tissue culture plates, pipette tips, dishes, plates, and other vessels that come into physical contact with gametes, embryos, or tissue culture media. As used herein, IVF consumables can include assisted reproduction water and water purification systems intended to generate high quality sterile, pyrogen-free water for reconstitution of media used for aspiration, incubation, transfer or storage of embryos for IVF or other assisted reproduction procedures as well as for use as the final rinse for labware or other assisted reproduction devices which will contact the embryos. In some instances, the product comprises needles, catheters, microtools, labware, syringes, tissue culture dishes, tissue culture plates, pipette tips, dishes, plates, water, water purification systems, media, media supplements, or other devises or reagents that come into physical contact with embryos.

In some embodiments, the method for assessing a product used for assisted reproductive technologies (ART) can reduce morphology-based embryo grading variability. In some instances, the method can enable visualization of the nuclear localization of the fusion protein, optionally after 48 hours post embryo culturing. In some cases, the method can reduce false positives compare to an equivalent assay, such as the mouse embryo assay (MEA).

In some embodiments, the product is a protein or a gene associated with a disease. The product can also encompass the transgenic mouse comprising the protein or gene for use as a murine model. The disease can be a cancer. In some cases, the cancer is a solid tumor. In other cases, the cancer is a hematologic malignancy. The protein or gene can be associated with a cancer, optionally associated with a solid tumor or a hematologic malignancy. The protein or gene can be a tumor associated antigen. Exemplary tumor associated antigens include, but are not limited to, CD19; CD20; CD22 (Siglec 2); CD37; CD 123; CD22; CD30; CD 171; CS-1; epidermal growth factor receptor (EGFR); epidermal growth factor receptor variant III (EGFRvIII); human epidermal growth factor receptor (HER1); ganglioside G2 (GD2); TNF receptor family member B cell maturation (BCMA); prostate-specific membrane antigen (PSMA); Receptor tyrosine kinase-like orphan receptor 1 (ROR1); Fms-Like Tyrosine Kinase 3 (FLT3); or Tumor-associated glycoprotein 72 (TAG72). The protein or gene can also be an overexpressed or repressed protein or gene in a cancer subject, compared to the expression of the protein or gene in a normal subject.

In some instances, the product is a protein or a gene associated with an autoimmune disease, and/or the transgenic mouse comprising the protein or gene for use as a murine model. The protein or gene can be overexpressed or repressed in a subject suffering the autoimmune disease, compared to the expression of the protein or gene in a normal subject.

In some embodiments, the product is a protein or a gene associated with the development of the embryo. The protein or gene can be associated with regulating protein-protein interaction(s) or gene expression(s), metabolic processes, cell morphogenesis, cell division, cell proliferation, DNA replication, cell differentiation, or DNA repair and transcription. The protein or gene can be associated with cellular communication, apoptosis, immune response, housekeeping, or tissue specific functions. Exemplary proteins or genes can include, but are not limited to, pluripotent stem cell (PS)-specific markers such as the family of Sox genes (e.g., Sox1, Sox2, Sox3, Sox15, and Sox18); the family of Klf genes such as Klf4 and Klf5; or the family of Nanog genes such as NANOG; markers associated with the TGF-beta superfamily and their respective receptors; markers associated with the cryptic protein family (e.g., Cripto-1); markers associated with the integrin family (e.g., integrin alpha 6 (CD49f) and integrin beta 1 (CD29)); markers associated with the Podocalyxin family (PODX-1), the FGF family (e.g., FGF4 and FGF-5), the Forkhead box transcription factor family (e.g., FoxD3), the T-box family of transcription factor (e.g., TBX3 and TBX5), the family of developmental pluripotency associated molecules (e.g., Dppa2, Dppa3/Stella, Dppa4 and Dppa5/ESG1), the LRR family (e.g., 5T4), the cadherin family (e.g., E-Cadherin), the connexin family of transmembrane proteins (e.g., Connexin-43 and Connexin-45), the F-box family of “other” category (e.g., FBOXO15), the family of chemokine/chemokine receptors (e.g., CCR4 and CXCR4), or the ATP-binding Casstet Transporters (e.g., ABCG2).

In some embodiments, one or more embryonic stem cells are further obtained from the transgenic embryo. The one or more embryonic stem cells can be cultured to generate a plurality of embryonic stem cells. The plurality of embryonic stem cells can be subsequently cultured with a drug. The expression of the fusion protein can be evaluated to determine acceptability or failure of the drug. In some cases, the drug is for use in the treatment of a disease, optionally a cancer or an autoimmune disease. In some cases, the drug is for use in modulating an immune response.

Qualitative analysis of embryo development can be accomplished by analyzing the developing embryo by assessing the color, light intensity or fluorescence visually, e.g., with a light microscopy which may include UV light to visualize fluorescent protein expression. A confocal microscopy may also be utilized for assessing the developing embryo. In some cases, embryonic development is observed via an embryo scope (e.g., EmbryoScope® Time-lapse system, Unisense Fertilitech A/S), wherein a picture of developing embryos can be taken as desired, for example, approximately every 5, 10, 20, 30, or more minutes and a time-lapse video can be generated to track all stages of embryo development.

### Kits and Articles of Manufacture

In certain embodiments, the present disclosure provides kits for performing the methods of this disclosure as well as instructions for carrying out the methods of the present disclosure. The kit comprises, or alternatively consists essentially of, or yet further consists of one or more of: constructs for introducing the fusion protein described above, modified eggs (e.g., oocytes and/or zygote), transgenic embryo, and/or the transgenic mouse described above, and instructions for use.

The kit can also include culture media and/or supplements, for use with the methods of this disclosure. In some instances, the culture media includes, without limitation, reproductive media and supplements used for assisted reproduction procedures. Media can include liquid and powder versions of various substances which come in direct physical contact with embryos (e.g. water, acid solutions used to treat gametes or embryos, rinsing solutions, reagents, sperm separation media, or oil used to cover the media) for the purposes of preparation, maintenance, transfer or storage. Supplements can include specific reagents added to media to enhance specific properties of the media such as proteins, sera, antibiotics, or the like. As amenable, these suggested kit components may be packaged in a manner customary for use by those of skill in the art.

### Partial Sequence Listing

| OCT4 protein sequence- | |
|---|---|
| SEQ ID NO: 1 | |
| AGHLASDFAFSPPPGGGDGSAGLEPGWVDPRTWLSFQGPPGGPGIGPGS | |
| EVLGISPCPPAYEFCGGMAYCGPQVGLGLVPQVGVETLQPEGQAGARVESNSEGTS | |
| SEPCADRPNAVKLEKVEPTPEESQDMKALQKELEQFAKLLKQKRITLGYTQADVGL | |
| TLGVLFGKVFSQTTICRFEALQLSLKNMCKLRPLLEKWVEEADNNENLQEICKSETL | |
| VQARKRKRTSIENRVRWSLETMFLKCPKPSLQQITHIANQLGLEKDVVRVWFCNRR | |
| QKGKRSSIEYSQREEYEATGTPFPGGAVSFPLPPGPHFGTPGYGSPHFTTLYSVPFPE | |
| GEAFPSVPVTALGSPMHSN | |
| GFP protein sequence- | |
| SEQ ID NO: 2 | |
| MVSKGEELFTGVVPILVELDGDVNGHKFSVSGEGEGDATYGKLTLKFIC | |
| TTGKLPVPWPTLVTTLTYGVQCFSRYPDHMKQHDFFKSAMPEGYVQERTIFFKDD | |
| GNYKTRAEVKFEGDTLVNRIELKGIDFKEDGNILGHKLEYNYNSHNVYIMADKQK | |
| NGIKVNFKIRHNIEDGSVQLADHYQQNTPIGDGPVLLPDNHYLSTQSKLSKDPNEKR | |
| DHMVLLEFVTAAGITLGMDELYK | |
| OCT4::EGFP fusion protein sequence- | |
| SEQ ID NO: 4 | |
| MAGHLASDFAFSPPPGGGDGSAGLEPGWVDPRTWLSFQGPPGGPGIGPG | |
| SEVLGISPCPPAYEFCGGMAYCGPQVGLGLVPQVGVETLQPEGQAGARVESNSEGT | |
| SSEPCADRPNAVKLEKVEPTPEESQDMKALQKELEQFAKLLKQKRITLGYTQADVG | |
| LTLGVLFGKVFSQTTICRFEALQLSLKNMCKLRPLLEKWVEEADNNENLQEICKSET | |
| LVQRKRKRTSIENRVRWSLETMFLKCPKPSLQQITHIANQLGLEKDVVRVWFCNRR | |
| QKGKRSSIEYSQREEYEATGTPFPGGAVSFPLPPGPHFGTPGYGSPHFTTLYSVPFPE | |
| GEAFPSVPVTALGSPMHSNSGGGGSGGGGSGGGGSMVSKGEELFTGVVPILVELDG | |
| DVNGHKFSVSGEGEGDATYGKLTLKFICTTGKLPVPWPTLVTTLTYGVQCFSRYPD | |
| HMKQHDFFKSAMPEGYVQERTIFFKDDGNYKTRAEVKFEGDTLVNRIELKGIDFKE | |
| DGNILGHKLEYNYNSHNVYIMADKQKNGIKVNFKIRHNIEDGSVQLADHYQQNTPI | |
| GDGPVLLPDNHYLSTQSKLSKDPNEKRDHMVLLEFVTAAGITLGMDELYK | |
| Construct sequence- | |
| SEQ ID NO: 5 | |
| 1 | gggcctacta agtgttcact atgcagcagg ggctagcctg aactcccaga tagcattact |
| 61 | gttatatata aataatagca ttactgttat tttaaattta gtttgttatg tgtatgaggg |
| 121 | ttttgctttc ctgtgagcat gccctcaaag gtcagatgag ggcattggat cctctggaac |
| 181 | tgaagttcca aatgattgtg agccatccat cattcaggtg ctgagaactg agctcaggtc |
| 241 | ttctccaaga gcagcaggtg ctatttaaat tctctctcgc cgggcgtggt ggcgcacgtc |
| 301 | tttaatccca gcactctgga ggcagaggca ggaggatttc tgagttcgag gccagtcctc |
| 361 | tccctcttct tcttcttctt ttttttttcc gagacagggt ttctctgtgt agccctggct |
| 421 | gtcctggaac tcactctgta gaccaggctg gcctcgaact cagaaatttg cctgcctctg |
| 481 | cctcccaagt gctgggatta taaattcttt ttttttttta agatttattt atttattata |
| 541 | tataagtaca ctgtagttgt ctccagacgc accagaagag ggcgtcagat ctcattacgg |
| 601 | gtggttgtga gccaccatgt ggttgctggg atttgaactt cggacctttg gaagagcagt |
| 661 | cgggtgctct tacccactga gccatctcac cagcccctgg attataaatt cttatttgta |
| 721 | cagtttttgt tgttggtttt gtttgtttgt ttgtttcaac acttgggatt gaatccaggg |
| 781 | tcattggagt gtttggcctg ggttctgctg ctgagccaca ctccatatac ttgcatggat |
| 841 | tgtgtctttt attctatgtg gtggtggtgg tggtggtggt ggtggtggtg gtggtggtgg |
| 901 | tggtggtggt agagggaact cacccagccc acctcctctg gtttcactcc tccacacaca |
| 961 | cccttcatcc ctgtccttac cccctgccag taagaaactc cacaggatgt ctctctagga |
| 1021 | gtctgagaaa aaagcactaa gagaacaagc tgtttgtttt ttggggtttt ttttgggggg |
| 1081 | gtgggggggg gagttagaac tcaggaaatc agaacagggc taggtgtggt aatgaatgac |
| 1141 | atagatctag tcactcaaag gctgtgtacc tctgaagttg agactagcct ggtttatata |
| 1201 | aaaagtaacc tggtctatat aaaaaattct aagttacgcc gggcgatgat ggcacatgcc |
| 1261 | tttgatccta gcacttggga ggcagaggca ggcggatttc gaagtttgag gccagcctgg |
| 1321 | tctacaaagt gagttccagg acagccaggg ctacacagag aaaccctgcc ttgaaaacaa |
| 1381 | acaaacaaac aaacaaaaaa ccaaaaacaa ccaaaacaaa caaacaaaaa aaccccaaaa |
| 1441 | ccaaaacaaa acaaaacaaa aagttctaag ctatgtagaa ctcttggggc actcgtgatc |
| 1501 | aaagtctaag cattgaaaat ctctaaatgc caggcttggt ggcgcacgca cttaatacca |
| 1561 | gcacttggga ggcagaggca ggcagatttc tgagttcgag gccaacctgg tctacaaagt |
| 1621 | gagttccatg acagccaggg ctacacagag aaaccctgtc tcgaaagaca aaaaaaaaaa |
| 1681 | aaaaaaaaaa aaaaaaaaat ctctaaacaa gccctatcaa tcttcccttg ggggtatgga |
| 1741 | ctgttatctc ttcatcataa gaggaaagcg aataggtggt gtgaaggagg tctttggtgg |
| 1801 | tgcaggaggg agccactggc tgcaggggac acagggtaca gcatggctcc ttagttggac |
| 1861 | atctgcttga cagaatgagt gtctatctat ctgtggactt gggaatcaac cccaggtatt |
| 1921 | ccaggaggta agaagaacca ctctctgaac cagggtggtc ttttgagcca ccagaaaaaa |
| 1981 | catccaccat gtaattgtaa ccaagtaacc agtcataacc ccctgcaaca gcgcccagga |
| 2041 | gacttttgaa tctgaaacaa gcagaaccac agagaggggg aggaaaggtt atcctgaccc |
| 2101 | agagaggcaa atcctctctg gaaccccggg agctaggtca gatgctaagg gcatttgtgg |
| 2161 | ggagggctgg gagagaaggg ccctaggcta gcagcaggtc ctccttccct ttaacctctg |
| 2221 | gccatgttgg gtagggactc tccttgacaa acctgataac caggagttca tttccgagca |
| 2281 | cccacacagt ggaagaagct agcccatcgc ctaaagttgc ccttttgttc tctttttctc |
| 2341 | tctctcacac aaatttttaa attttattat ttatttattg acataatttc tctatgtatc |
| 2401 | ccaggctgtc tcagaactca ctttgtagac caggctggcc tcaaacttag agatgctcct |
| 2461 | acctctgcct cctgagtact aggattaagg atatccatca caacacctaa ctgtaataaa |
| 2521 | aatttcaatt tttttttctc tcttagcctc agctgcaatc acctctttta gccagtatac |
| 2581 | ctgcaggagc tccagtccat aggcgggctc tcctaatgcc tgaatttgac tctcaaagtg |
| 2641 | cattcacttc ctctcattac agctcttctg cagactcttt tatatcagga atctcaaaac |
| 2701 | ttgggttggg catggtactc gcctttaatc ccaggaggtg gaggccagcc tggtgtacaa |
| 2761 | agtgagttct aggtcaggca ggactgtaat gtctactcct tacatgaaca ggatagggcc |
| 2821 | tggcggggag agagggggga agctaggtgg gagtaagggt ggaaacgggc aatcgtgtca |
| 2881 | atggaagaag taaatgatca agacaatagg tctgttcaca gcattagaac cacccaaatg |
| 2941 | gtcatttggc atcggagtcc tctccctcct cccaatcccc ccccccattt gcttatctat |
| 3001 | ttatttttat tttatgtatg caagcatttt ttttttttct ttttctttct gggtttttcg |
| 3061 | agacagggtt tctctgtgta gccctggctg tcctggaact cactctgtag accaggctag |
| 3121 | cttcgaactc agaaatctgc ctgcctctgc ctcccaagtg ctggaatcaa aggcaagggc |
| 3181 | caccacacct ggcgcaagca tttttcaaag atgtatttgt ttatatatgt gagtacatta |
| 3241 | tctctctctt caggcacacc aggagagaga gagagagaga gagagagaga gagagagaga |
| 3301 | gagagagaga gagagagaga atcagatcct gtcacagatg gttgtgagtc accaagtggg |
| 3361 | agctgggatt tgaactcagg acctctggaa gagctgtcag tgcacttaac ctgtgagcca |
| 3421 | tctctccagt ccttttcctc tctgtttttt tttttttttt tttttttttt ttgacacagt |
| 3481 | ttaaccatgt agcttaggct aatcttcagt ttgctgtgcc tcagcttctg agtgtccact |
| 3541 | tgggattgca ggagtttgcc gccttgctca gcttacagct tgggatgctc ttctcacaga |
| 3601 | catccacgta ctagtactga aagacaaaga caaacaccgg tttgcttgca aactgctcaa |
| 3661 | gtcttgtcta gtttgattag gcttgagagg agaccttgcc agctttccca agaagactgt |
| 3721 | gtggaggttg gcggtagaag gtggggtaca gaagacttgc tttcatctca caaggtgccc |
| 3781 | cacccatctc tctaacgccc aatttttctt tacagaggca cagggacaac tccctacccc |
| 3841 | gacccatgtg gagtgagaag ggcaggagga tgcatgggaa tagggctgta gttttctttt |
| 3901 | caagttggaa gaaaatggag agttaggcca gggaaggtag tgacaccccc ctggggtgtc |
| 3961 | acacacacac acacacacac acacacacac acacacacat acacacacag ggaattctag |
| 4021 | cactggtggt gtgagcaagt aggtagctat atgtagtaat ataacaagat ttattttgct |
| 4081 | gagagtcatc acataaggtt cacactctaa gccagccagg tggtggtggt ggcacatgcc |
| 4141 | tttaatccca gcacttcgga gacagagaca ggaggatttc tgagttcgag gccagcctgg |
| 4201 | tctacaaagt gagttccagg atagccaggg ctacacagag aaaccctgtc tcgagagaaa |
| 4261 | aaaaaaaaaa aaaggttcac actctgtaaa ttcctaacta atgtctcatt cctgtctccc |
| 4321 | cacagctgct cacctatgtg acatatttta gcagaaggtc aggtccactc tcttaatctc |
| 4381 | ttaagagtgt ctgtgatttg agggacagga tcctagcggg gagactctcc acctacaagg |
| 4441 | cagatcaagg tggctggtta tcttgaatga gtgatgtcgt ggggttctgg ttatcagggg |
| 4501 | cagccttggg tgtagtggtg aagccatttc tgcaagtcta aaaggcattt ggacactgct |
| 4561 | catcagaact gggaaggcaa gaggatcaga agttcaaggg ccttcttggc tacgtagagt |
| 4621 | ttaggggcag cctctgctac atgtaaattt gtctcagaag aaaaaaggaa acgggcagta |
| 4681 | gtggcgaatg cctataatcc cagcacttgg gaagcagagg gcaggctgat ttctgagctc |
| 4741 | aaggccagcc tggtctacag agtgagttcc aggacagcca gggctacaca gagaaaccct |
| 4801 | gtctcagaaa gaaaaaaaaa aaaagggaga aatagaactc atttatttca aagggagcag |
| 4861 | gcacattccg caagccttct gttctgctgg tgacctattc ctaccttcag cttctagatc |
| 4921 | tttgtttttt cttttaattt ttattgtatt gtgtgtgtgt gtgtatctga gtgcaatatc |
| 4981 | aatatcgaac ctctggagat caaatgcttc tttgaaatgg accccaggga tcaaatccat |
| 5041 | ggtcgttagg ctcagcaagt gtggttactt gctgagctgt cctcaggttc ctctcaggcc |
| 5101 | tcccctcaag tctccccctc caaccccccc cccctcctgc cccctgcaag tctttttgat |
| 5161 | taggcaagaa aaccaagaca aggaaaggga gatgcagtta gctaaggaat ctatgccagc |
| 5221 | cagagaaact acctcctccc ttccagaaca tctggatttg ggaagagacg ttgctggtcc |
| 5281 | cagggcggct gggggttggg gttgggggag ggggatgcta accagcaagg aagctgttcc |
| 5341 | tggctggggc aggcctgact gagctcatgt cgctgaaact cctcatttct ccctatggct |
| 5401 | tcatagggag acccagcctg gatgctaaca cgagtgattt ccctgctcta gtctagtgtc |
| 5461 | ctccgtgagt ccatttaact gatcacccag tctgtgagga ggtggctgaa ctcacagtaa |
| 5521 | gaaagctgtg ggggtcaacg cctattgttt gtttgttttg ttttagacaa ggtctcctgc |
| 5581 | tgaggctggc tcaagctggc ctggaggact cttgtgttta aggctggcct taaattctct |
| 5641 | ttaaaagaaa atcatgtgta tatatgtgtg tccatgtaac tgcagatgac cacagcagcc |
| 5701 | aagagatttc tgttctccta gctgtaaacc acccaatatg ggtgctagga acagaatttt |
| 5761 | aaaagggtcc tctgaaagag caatgtacac tcaaatgctg agttctttcc cagcccctag |
| 5821 | ccttggacct ttgttcttat cacttccaac gcccaagggc aggcattata ggtgtggcat |
| 5881 | tccgcatctg gcttcccagg ataccttttc atgctggtgg accatctctg gctggggacg |
| 5941 | tgtgggcttc tctgctgtct ttggttctcc agacagaact ccgagacaga tcttgacttg |
| 6001 | gttctaaaat acaggtggtt tgtggcaagt taacgaattt tagctcaaat ttggggtatt |
| 6061 | taagatacca tggtgactct tttttgtttg tttttctttc tttctttttt ttttttaaaa |
| 6121 | agatttattt attattgtac gtaagtactt cagacaccag aagagggtga cagatttttt |
| 6181 | ttttttttcg agacaggatt tctctgtgaa gccctggctg tcctggaact cactctgtag |
| 6241 | accaggctgg cctggaactc agaaatccac ctgcctctgc ctcccaagtg ctgggattaa |
| 6301 | agccatgagt tgggaccgca cccggcccaa agtgactctt aaagggggca gagtggcaca |
| 6361 | tattttcaat cctagcactc aggaagcaaa ggctggtgga tctctgtgag ttcaaggcca |
| 6421 | gcctggtcta cagagtgagt tccaagccat ctaaggctat gtagggaacc cttgaatcaa |
| 6481 | acccaaaagt tagtctgatg attttctaag acccaggagg caagaaactg gatcagatga |
| 6541 | gccaacaggt ctgctgtccc atctccaggg ccaccaggct cacagctcgg gaccaggcta |
| 6601 | gggcacatct gtttcaagct agttctaaga agacttggga cttcagacaa agttgctgtt |
| 6661 | aaggactgta ttatactcta ggcacgctta gggctaacct ggttgcaaag ccagtcacta |
| 6721 | ggcagttaaa ggactcagaa tatgtctctt gtcctggcca gtgagtcacc aaaagagaaa |
| 6781 | tcacaatcca taagacaagg ttggtattga atacagacag gactgctggg ctgcaggcat |
| 6841 | acttgaactg tggtggagag tgctgtctag gccttagagg ctggccctgg gaggaactgg |
| 6901 | gtgtggggag gttgtagccc gaccctgccc ctccccccag ggaggttgag agttctgggc |
| 6961 | agacggcaga tgcataacaa aggtgcatga tagctctgcc ctgggggcag agaagatggt |
| 7021 | tgggggaggg tccctctcgt cctagccctt ccttaatctg ctattgagga agctttgtga |
| 7081 | acttggcggc ttccaagtcg ctgcctttat ttaggtcttc caactaacct atggcactgt |
| 7141 | tccacaatga atgtatagaa attgggaggt gagcatgaca gagtggagga aacggaagat |
| 7201 | tcatggagag ggccagagag atggcccctc agccaccctg ggggatgact tggacccatg |
| 7261 | tggtagaagg aggggacttc cacacatgtg ctatgtgtag ctgtgtgtag gtacatacac |
| 7321 | acccttaaaa taaaacgcaa tttttttttc aaagtctcag ggtgaatttg gtgaagtcga |
| 7381 | tgaagctgag gcaggagaat tatcaggagt tcaagggcag cttgttttat agagaaaggt |
| 7441 | tccatctcta cctgatgaag actaccatca agagacaccc ccgcccccca gggcacctag |
| 7501 | agccactgac cctagccaac agctcaggcg ggctgggccc aggctcagaa ctctgtcctg |
| 7561 | gctatgtaca ctgtggggtg ctctgggctt tttgaggctg tgtgattcac cctggggcct |
| 7621 | tcgttcagag catggtgtag gagcagacag acaaacacca tcccttgcag acaggcactc |
| 7681 | tgagggctat tctcttgcaa agataactaa gcaccaggcc agtaatggga tcgtgaccca |
| 7741 | aggcaggggt gagaggacct tgaaggttga aaatgaaggc ctcctggggt cccgtcctaa |
| 7801 | gggttgtcct gtccagacgt ccccaacctc cgtctggaag acacaggcag atagcgctcg |
| 7861 | cctcagtttc tcccaccccc acagctctgc tcctccaccc acccaggggg cggggccaga |
| 7921 | ggtcaaggct agagggtggg attggggagg gagaggtgaa accgtcccta ggtgagccgt |
| 7981 | ctttccacca ggcccccggc tcggggtgcc caccttcccc atggctggac acctggcttc |
| 8041 | agacttcgcc ttctcacccc caccaggtgg gggtgatggg tcagcagggc tggagccggg |
| 8101 | ctgggtggat cctcgaacct ggctaagctt ccaagggcct ccaggtgggc ctggaatcgg |
| 8161 | accaggctca gaggtattgg ggatctcccc atgtccgccc gcatacgagt tctgcggagg |
| 8221 | gatggcatac tgtggacctc aggttggact gggcctagtc ccccaagttg gcgtggagac |
| 8281 | tttgcagcct gagggccagg caggagcacg agtggaaagc aactcagagg gaacctcctc |
| 8341 | tgagccctgt gccgaccgcc ccaatgccgt gaagttggag aaggtggaac caactcccga |
| 8401 | ggaggtaagt gaagggactt ggctgggctg gcagaggcag cagtgaaggg aattgggaac |
| 8461 | atgtagggta gccaccctgc ctgccaaagg tggtgatggc tgccgggcct cctgagaagc |
| 8521 | acgacgcagt gtggactaga acccagaatt gcaagaatca gaaaccggcc tggattgttt |
| 8581 | cggcctggcc cttgtcatgt aggtcaccta ggcctggcct gtgtcccgac acttgcttca |
| 8641 | tgccatcact gtctgtacac cagtgatgcg tgaaaatcag ccccccccca aaaaaaaaaa |
| 8701 | catatcagcc cctctgggga cttggatcac agtcggaccc aggaacttgg ccttaaggtt |
| 8761 | aggcatggct gggggggtaa aaaatggtgc ttatcctgga gttattgtta ctgaagaggt |
| 8821 | tgggtgtgac tggctgctga taggagctct tgtttgggcc atgtgtggag tagggctcac |
| 8881 | cttcagtcaa gtttacggcc tgtctacttt agcctcagac tccatgagtc acctttacac |
| 8941 | gagcagaccc ttgtagtgcc tgaggtgcag atctgatcga tttcagcctt tctacctttc |
| 9001 | cttgtaaaca agaaagggac acccttgggt aggggagttt tatctccagg ccatcttaag |
| 9061 | atcattctgt gagtgcacgg gccttgctta gtgtctgatg gcctacagcc agcactctgg |
| 9121 | agcaagtgta agcaattagc cttaagaaca aggtgcgagt ggataccgat gcccgccggg |
| 9181 | agttccgaca gcttagcgat tgttgtagca ggagtcccct ccctaagtgc cagtttctgt |
| 9241 | gttatctcag gtcctgtatg ccgccgggag tcccctagga aggcattaat agtttatctc |
| 9301 | acatcttaaa tggcccttaa tgaagcaaga gatttgaacc ttagttaagc taatcccaaa |
| 9361 | tcctcaaaat aggatttaga aaagccaaag acactgctga gggcgattac aagttttggt |
| 9421 | cttttgagga gcagttggag atgaaagtct gtctgaagcc gagagaatcc ttttccattg |
| 9481 | aaatggcatt gaggtgtgcc tcactggctg ctgcttctgt ctgtgccctg ggttggccag |
| 9541 | cctttgtgga gcacctcagc cctccatcct ggacctttgc tccaacaacc tgctcctctt |
| 9601 | ccgccctcaa ggctgacttg catctcccca gatgactgcc tccatttctg tcttctgtta |
| 9661 | gagacagaaa agcctgagaa accgacagcc attttggggg gggggggtcc ggttcacacg |
| 9721 | ctgcaactta gaaagcacac tcaactggcc atctgttata ccctccccac ctggtcccaa |
| 9781 | ccatcactgt gtactactga gaagaaggca gccttagcca caccctcgag tgcccctgcc |
| 9841 | gttctattgc tcatacatcg attgatatcc ctgtttcaac tttgaaaaaa aaaaattttt |
| 9901 | ttttttttgt ggtgtgtgca tgcctgctac tgtacacctg tgggcgtcag aggtggtcct |
| 9961 | ctgcaccctc cggccagtac cgcatccagg gtgagtcaga tgatttcctg tggtttgggc |
| 10021 | ctcaaggctt ctcacctcca gaggcttcta gcctgctgcc ttgctttctc tgtcgcactc |
| 10081 | tagtacagca ggagttttct tcgcactccg gagtgttgtc agctcctggg gcatggacat |
| 10141 | ttggctactt agagtgtgct gtgtaggttt tcatttagag ctgaacagag ggatggatct |
| 10201 | tattacccca gcccttgaga cactgaggca ggagagcttc ctagtgagtc cctgtttcaa |
| 10261 | tatcttcact aatactgtgt catactttgg gactttcttt cttcctttct ttcttttgat |
| 10321 | tttttttttt tttatatgag tacagtgtac ctgtcttcag acacacacca gaagagggca |
| 10381 | tcagatccta ctacagaagg ttgtgagcca ccttgtggtt gctgggaatt gaactccgga |
| 10441 | tctccggaag agaagtccgt ataccaactt ctgtattagt cagggttctc tagagtcaca |
| 10501 | gaacttatgg acagtctcta gatagtaaag gaatttattg atgacttaca gtcggcagcc |
| 10561 | caattcccaa caatggttca gtcgcagctg tgaatggaag tccaaggatc tagcagttac |
| 10621 | ttagtctcac gcagcaagca ggcgaaggag caagagctag agcttaactg ctgagccatg |
| 10681 | tgtttcttga gtaaagggat tacatgctcg ttcgtctggt caattctgca gccttaaaac |
| 10741 | ttcttcagaa tagggtgaca ttttgtcctc agtggggcgg ttttgagtaa tctgtgagca |
| 10801 | gataggaact tgctggggta ctgcacagaa ctctgggtag tgtggtactg tagatggcta |
| 10861 | ggttctgggg ggggaaagag ccatctatgt cacctaggaa tagagtgaat aacatttata |
| 10921 | taatcagacc agcccttgag gaggctgaga tcttttcatg gggcacccta gggtcacagt |
| 10981 | cccagctggt gtgactctga caagtctgcc tttctcacta cagtcccagg acatgaaagc |
| 11041 | cctgcagaag gagctagaac agtttgccaa gctgctgaag cagaagagga tcaccttggg |
| 11101 | gtacacccag gccgacgtgg ggctcaccct gggcgttctc tttggtgggt ctcccccagc |
| 11161 | atgttctgat ctcacggctc ttaatgtagg cgcaaggggg tggggcatct taggagctgc |
| 11221 | ttctccacag gtaagggagg attagacgct tgtagcttga actgtcagag gtgggggctt |
| 11281 | gggctccctt cttgctgcct cactcactct gtttgatcgg cctttcagga aaggtgttca |
| 11341 | gccagaccac catctgtcgc ttcgaggcct tgcagctcag ccttaagaac atgtgtaagc |
| 11401 | tgcggcccct gctggagaag tgggtggagg aagccgacaa caatgagaac cttcaggagg |
| 11461 | tgaggagtgg caggatgtgt gcaatgtctg ccaggcacag tcccttctgc tgcttccatt |
| 11521 | cctggcttga aactcctccc tctccaaccg gagctcgcag gagaagttct gtgtccttat |
| 11581 | tctgctgcta tgaattggaa tccagagcct taacatttgc taatcaatca ggctctctcc |
| 11641 | ttctgagtca ccctctgccc ccaccagcct gacaatggtc cctccccaga accccgtcta |
| 11701 | gtgctggtga aggctcagac ctaggtctac cagccccttc cagagcccct ttcagtaacc |
| 11761 | cctggctctg gggccacatc cagtcaatgc tcccttagca caatccctta gcggtttgtt |
| 11821 | cttcagtccc atctcaaggt ggggctgttg ccaagtcaaa tactaaagtt gctcttgtcg |
| 11881 | cccccatctt cccctgccca gatatgcaaa tcggagaccc tggtgcaggc ccggaagaga |
| 11941 | aagcgaacta gcattgagaa ccgtgtgagg tggagtctgg agaccatgtt tctgaagtgc |
| 12001 | ccgaagccct ccctacagca gatcactcac atcgccaatc agcttgggct agagaaggat |
| 12061 | gtgagtgcca agatcctgcc ctgtggtacc tggatgtttc cctgttccca ttccccaccc |
| 12121 | cccccacccc cccaccccca ccgccgccac cgctgactgc agcatcccag agcttatgat |
| 12181 | ctgatgtcca tctctgtgcc catcctaggt ggttcgagta tggttctgta accggcgcca |
| 12241 | gaagggcaaa agatcaagta ttgagtattc ccaacgagaa gagtatgagg ctacagggac |
| 12301 | acctttccca gggggggctg tatcctttcc tctgccccca ggtccccact ttggcacccc |
| 12361 | aggctatgga agcccccact tcaccacact ctactcagtc ccttttcctg agggcgaggc |
| 12421 | ctttccctct gttcccgtca ctgctctggg ctctcccatg cattcaaact ctggcggagg |
| 12481 | cgggagcggg gggggtggct ccggcggcgg agggagcatg gtgagcaagg gcgaggagct |
| 12541 | gttcaccggg gtggtgccca tcctggtcga gctggacggc gacgtaaacg gccacaagtt |
| 12601 | cagcgtgtcc ggcgagggcg agggcgatgc cacctacggc aagctgaccc tgaagttcat |
| 12661 | ctgcaccacc ggcaagctgc ccgtgccctg gcccaccctc gtgaccaccc tgacctacgg |
| 12721 | cgtgcagtgc ttcagccgct accccgacca catgaagcag cacgacttct tcaagtccgc |
| 12781 | catgcccgaa ggctacgtcc aggagcgcac catcttcttc aaggacgacg gcaactacaa |
| 12841 | gacccgcgcc gaggtgaagt tcgagggcga caccctggtg aaccgcatcg agctgaaggg |
| 12901 | catcgacttc aaggaggacg gcaacatcct ggggcacaag ctggagtaca actacaacag |
| 12961 | ccacaacgtc tatatcatgg ccgacaagca gaagaacggc atcaaggtga acttcaagat |
| 13021 | ccgccacaac atcgaggacg gcagcgtgca gctcgccgac cactaccagc agaacacccc |
| 13081 | catcggcgac ggccccgtgc tgctgcccga caaccactac ctgagcaccc agtccaagct |
| 13141 | tagcaaagac cccaacgaga agcgcgatca catggtcctg ctggagttcg tgaccgccgc |
| 13201 | cgggatcact ctcggcatgg acgagctgta caagtaatga ggcaccagcc ctccctgggg |
| 13261 | atgctgtgag ccaaggcaag ggaggtagac aagagaacct ggagctttgg ggttaaattc |
| 13321 | ttttactgag gagggattaa aagcacaaca ggggtggggg gtgggatggg gaaagaagct |
| 13381 | cagtgatgct gttgatcagg agcctggcct gtctgtcact catcattttg ttcttaaata |
| 13441 | aagactggga cacacagtag atagctgaat tttgttttcc ttcagttcct agagagcctg |
| 13501 | cggttggaga aagccagtaa tggattctca aaccccaggt gatcttcaaa acaggcgcca |
| 13561 | ttgaaaccat tggagttcca caaaatgccc agggatagtt ggggttggag cccaacctat |
| 13621 | agaggaaggc attgcatatt cgccatccta gaggcggtaa gtctctgcta gctgatggac |
| 13681 | atcacctcat agccattgtc tggcagccgc cttctttcct cttgtcactc tgggagttct |
| 13741 | ggtgggctta tactttaaaa aaaagagttt ttttgggggg gttaagattt attttattta |
| 13801 | tatgggtaca ctgtagctgt cttctagaca caccagaaga gggcatggga tcccattaca |
| 13861 | gatggttgtg agccaccatg tggttgctgg gaattgaact caggacctct ggaagagcag |
| 13921 | tcagtgctct taaccgctga gccatctctc cagccctcaa actctttttt ttcttttcct |
| 13981 | tcaagatgag ttctgtgtag tcctggcgga ccaggttggc ctcagatcag cctgcctctg |
| 14041 | cctccgcagt gctgagatta aaggcccgtg ccactctagg ctaaattgtt atgcttctat |
| 14101 | tctagctgat gaccaccttt tttgggcgta gtagtgctgg gagtagggtc tgtacacatg |
| 14161 | tctacaatgc cagaataggt caaaggcttt agatctcaag gaactggatt tatagagagt |
| 14221 | tgggagcagc catgtaggtt ctgagaacca aacctgggtc ctctgcaaga agagccattg |
| 14281 | gctttttgtt tttgtttgtt ttgagacatt tctcggtgta gccctggcta tctggaactc |
| 14341 | tgtaggccag gctgtccctg aactcagatc cagtctatcc atccctgcct tccaagagct |
| 14401 | gggattaagg tcatgtacca ccacaggcca gctagccata gctcctaact gctgaaccat |
| 14461 | ttatttattt atttatttta tttttttggt ttttcgagac agggtttctc tgtatagctc |
| 14521 | tggatgtcct ggaactcact ttgtaaacca gtctggcctc gaactcagaa atctgcttgc |
| 14581 | ctctgcctcc ccagtgagtg ctgggatcaa atgcgtgcgc caccactccc agcttaagtc |
| 14641 | tttatttttt aaaatgttat ttattttggg gctggtgaga tggctcagtg gttaagagca |
| 14701 | ctgactgctc tgccagaggt cccgagttca aatcccagca actacatggt ggctcacaac |
| 14761 | catctgtaat gagatctgac gccctcttct ggtatgtctg aagacaacta cagtgtactt |
| 14821 | acatatatat ataataaata aattaaaaaa aaaaaagaga ggaggagcca agcagctcct |
| 14881 | ttagaaaaaa aaaaagttat ttattttatg tatttgagct gtcttcagac acaccagaag |
| 14941 | agggcaatgg atcccattac agatggttgt gagccaccat ggttgatggg aattgaactc |
| 15001 | tggacctctg gcagagcagt cagtgctctt aaccgctgag ccatctctcc agcccccaaa |
| 15061 | gccaagtctt aaagcatttt tgctgctgaa tgtcagccct accagatctc tgcctccctc |
| 15121 | ccctcccctc cccccagtat ctcatgaaga ccaagctggc ctggacacag taagtatgtg |
| 15181 | catatttatg tgtgaagatt gtcacaatgt gaggaaaaaa gttggttccc tccatggtgt |
| 15241 | gggtcctgtg ggtcaagtcc aggtcgctag gcttggcagc aagtgccttg actcatagtc |
| 15301 | ttctcctgcc caactccatg cttggtttcc acgagcccct gtgctatgga gaattccatc |
| 15361 | tccaggcctc accaaatcta atctccccat cctttgaaaa gcagacttag attcaacgca |
| 15421 | agtggatgaa acagtttatt ctttatttgg gaataaagac taagctctga aaagctagtc |
| 15481 | ccagagactc agctggtggt gatactagct agcggtggca tgaggatgcc ttgggaatgt |
| 15541 | gctctgggtc cttcagggtg ctttagccga tgccattcaa gaacatgagt agggttaggg |
| 15601 | tattgtggca gagcacttgc ctggtatatg ctggcttcag caaaataaaa ccataccttc |
| 15661 | taggaatggt ttctgggacc ggtgctctaa ctgcaggtat cctggcatcc atggaggcaa |
| 15721 | ggctttattc cttgtgactg ggcttgtagc tcactggaaa cttggaggct gcaacatctt |
| 15781 | tggcaggaaa ccatcttttc tgtcacttca tttgcaagca ttctccagcc ttgagtcagt |
| 15841 | ctttagcaat ggacctttcc ctgtggtcat tccctttgga gaaagacatt cctcaaagtc |
| 15901 | catggtaact ttgaatgagt gttttgcatg tacacatgcg tgagtgtgca tgcgctctca |
| 15961 | cacacgcacg cacatgcaca cgcgtgcaca cacacacaca cacacacaca cacacacaca |
| 16021 | cacacacaca ctgcttcagc ccttaggagc cattcttcta ttattatgtt tgagtgctct |
| 16081 | gcctgaatgt gcacctgcag gccagaagag ggcatcagat cccttttaga gatggtcaca |
| 16141 | agccatcatg ttgttgctgg aaattgggac ttctggaaga gcagccagtg tacccttaat |
| 16201 | tgctgagcca tcttactgcc caagatacat tcttacactg tgcctgaccc tgagccacat |
| 16261 | ctgtgtcctg actgcaaagt caagatgcca ttatggcatc ctggatgcta tagccagtgc |
| 16321 | aggccagagg gtaccagatg tcacagccat cacaccaacc caggctctgc tctctagcaa |
| 16381 | aaagaagctg gacaggactc cctaagggag tgagtgttcc tgagaaaccc tttgagtaac |
| 16441 | ttgcctctgg gtaactggta gccagaacag gaggctaaga ctgggatata ggaacttgga |
| 16501 | gattagggat gttaagtaga gcatacgcat agcacaaaag atacttggct ttggatatga |
| 16561 | gctgttgacg ccttcaatcc atcacaactc ccattctgaa tgctctatcc cgactacatg |
| 16621 | agggatttga ggctagcctg gattacacag tgagaccttg tattaaaaaa gagttgggtg |
| 16681 | tctcctccag agaggatctg ggtttgaacc tcagcaccta catagtggct agcaattatc |
| 16741 | cctccagttc ccagagaacc cagtgccctc ttctggcttc tgccggtatt gcatgcaagt |
| 16801 | gtgataccca atcatgcagg caaacaaagc agccttgaat tgacctgctc tcctctagtt |
| 16861 | ttgagacagt gttagtatgg ttttttatgt atagtgctgg gactccaaac atgggcaagt |
| 16921 | caggtgcttg ctaggcagtg ctcttctagt gagacatctc tttgttccct gtctcccaga |
| 16981 | ttgctttgta tagtctagtc ctaaccattg ttcccacata gtagcttgtc atgcatttat |
| 17041 | gggtgaaagc taacctgggt gtctgctgtg cccgtgcacc ccccttccct gccttctaag |
| 17101 | acctcagtct gaggctgttc aaagatctag aattcaaggt gctgacaggt gaccccactt |
| 17161 | acccactggc tatcagagca gctctggcga aaatgagacg ttggcgatcg cgtggcactt |
| 17221 | ttcggggaaa tgtgcgcgga acccctattt gtttattttt ctaaatacat tcaaatatgt |
| 17281 | atccgctcat gagacaataa ccctgataaa tgcttcaata atattgaaaa aggaagagta |
| 17341 | tgagtattca acatttccgt gtcgccctta ttcccttttt tgcggcattt tgccttcctg |
| 17401 | tttttgctca cccagaaacg ctggtgaaag taaaagatgc tgaagatcag ttgggtgcac |
| 17461 | gagtgggtta catcgaactg gatctcaaca gcggtaagat ccttgagagt tttcgccccg |
| 17521 | aagaacgttt tccaatgatg agcactttta aagttctgct atgtggcgcg gtattatccc |
| 17581 | gtattgacgc cgggcaagag caactcggtc gccgcataca ctattctcag aatgacttgg |
| 17641 | ttgagtactc accagtcaca gaaaagcatc ttacggatgg catgacagta agagaattat |
| 17701 | gcagtgctgc cataaccatg agtgataaca ctgcggccaa cttacttctg acaacgatcg |
| 17761 | gaggaccgaa ggagctaacc gcttttttgc acaacatggg ggatcatgta actcgccttg |
| 17821 | atcgttggga accggagctg aatgaagcca taccaaacga cgagcgtgac accacgatgc |
| 17881 | ctgtagcaat ggcaacaacg ttgcgcaaac tattaactgg cgaactactt actctagctt |
| 17941 | cccggcaaca attaatagac tggatggagg cggataaagt tgcaggacca cttctgcgct |
| 18001 | cggcccttcc ggctggctgg tttattgctg ataaatctgg agccggtgag cgtgggtctc |
| 18061 | gcggtatcat tgcagcactg gggccagatg gtaagccctc ccgtatcgta gttatctaca |
| 18121 | cgacggggag tcaggcaact atggatgaac gaaatagaca gatcgctgag ataggtgcct |
| 18181 | cactgattaa gcattggtaa tttgatatcg agctcgcttg gactcctgtt gatagatcca |
| 18241 | gtaatgacct cagaactcca tctggatttg ttcagaacgc tcggttgccg ccgggcgttt |
| 18301 | tttattggtg agaatccaag cactagtaac aacttatatc gtatggggct gacttcaggt |
| 18361 | gctacatttg aagagataaa ttgcactgaa atctagaaat attttatctg attaataaga |
| 18421 | tgatcttctt gagatcgttt tggtctgcgc gtaatctctt gctctgaaaa cgaaaaaacc |
| 18481 | gccttgcagg gcggtttttc gaaggttctc tgagctacca actctttgaa ccgaggtaac |
| 18541 | tggcttggag gagcgcagtc accaaaactt gtcctttcag tttagcctta accggcgcat |
| 18601 | gacttcaaga ctaactcctc taaatcaatt accagtggct gctgccagtg gtgcttttgc |
| 18661 | atgtctttcc gggttggact caagacgata gttaccggat aaggcgcagc ggtcggactg |
| 18721 | aacggggggt tcgtgcatac agtccagctt ggagcgaact gcctacccgg aactgagtgt |
| 18781 | caggcgtgga atgagacaaa cgcggccata acagcggaat gacaccggta aaccgaaagg |
| 18841 | caggaacagg agagcgcacg agggagccgc cagggggaaa cgcctggtat ctttatagtc |
| 18901 | ctgtcgggtt tcgccaccac tgatttgagc gtcagatttc gtgatgcttg tcaggggggc |
| 18961 | ggagcctatg gaaaaacggc tttgccgcgg ccctctcact tccctgttaa gtatcttcct |
| 19021 | ggcatcttcc aggaaatctc cgccccgttc gtaagccatt tccgctcgcc gcagtcgaac |
| 19081 | gaccgagcgt agcgagtcag tgagcgagga agcggaatat atccctaggt ataaacgcag |
| 19141 | aaaggcccac ccgaaggtga gccagtgtga ctctagtaga gagcgttcac cgacaaacaa |
| 19201 | cagataaaac gaaaggccca gtctttcgac tgagcctttc gttttatttg atgcgatcgc |
| 19261 | ctggagatcc ttactcga |

### EXAMPLES

These examples are provided for illustrative purposes only and not to limit the scope of the claims provided herein.

### Example 1-OCT4-GFP Transgenic Mouse Production

### Material and Methods

### BAC Cloning and Microinjection

A bacterial artificial chromosome (BAC) construct was used for expression of the Oct4 fusion protein. Monomeric EGFP was recombineered into the Oct4 (Pou5f1) locus. EGFP was inserted at the C-terminal of OCT4. To minimize steric hindrance between the reporter protein and OCT4, a flexible amino acid linker coding sequence (S(GGGGS)₃; SEQ ID NO: 3) was inserted between the gene coding sequence and the reporter gene. B6SJLF1 (Jackson Laboratory, Bar Harbor, ME) female egg donors were used. After sequential injection of PMSG (3 days before the harvest, at noon, 5 U per animal: Prospec, Rehovot, Israel, #HOR-272) and hCG hormones (1 day before the harvest, at noon, 5 U per animal, SIGMA, St. Louis MO, #CG5-1VL). The females were mated with B6SJLF1 males a day before the harvest. B6SJLF2 embryos were harvested at E0.5 and BAC construct for each transgene was injected into pronuclei. Injected embryos were implanted into the reproductive tract of pseudo-pregnant surrogate mothers (ICR: Charles River, Wilmington, MA). 20 days after the implantation, the number of newborn pups were counted and toe biopsy was performed at 7-10 days old to extract DNA for PCR genotyping.

### Genotyping

Two PCR methods were utilized for genotyping, conventional PCR and qPCR. For the conventional PCR, the annealing temperature was at 58° C. The following primers were used for detecting the eGFP sequence:

eGFP (product size = 227 bp)
TMF738 Forward:
(SEQ ID NO: 6)
5′-ATCTTCTTCAAGGACGACGGCAAC-3′
TMF739 Reverse:
(SEQ ID NO: 7)
5′-TCCTCGATGTTGTGGCGGATCTTG-3′
Internal Control (mouse Fndc3a gene: product size = 400 bp)
TMF725 Forward:
(SEQ ID NO: 8)
5′-GAGCTTCTGGTATTAGCGTTAGGT-3′
TMF726 Reverse:
(SEQ ID NO: 9)
5′-TCCACAATGACAAAGACATGAGGT-3′

Taqman qPCR protocol was used on a CFX-BioRAD qPCR set up. The EGFP transgene genotype was determined by comparing δCt values of EGFP against known homozygous (HO) and hemizygous (HEMI) controls and endogenous references (ApoB gene).

The following PCR condition was used: 95 deg C. 3 min->(95 deg C. 15 sec->60 deg C. 30 sec) time 40 cycles.

Table 1 illustrates the qPCR primers and probes used.

| Primer/Probe Sequence (all 5′→3′) | Target | Description |
|---|---|---|
| CCACATGAAGCAGCACGACTT | EGFP | For to TMF960 oIMR1856-modified genomic |
| (SEQ ID NO: 10) | | Probe (TMF961) qPCR. |
| GGTGCGCTCCTGGACGTA (SEQ | EGFP | Rev to TMF959 oIMR1857 genomic Probe |
| ID NO: 11) | | (TMF961) qPCR. |
| TTCAAGTCCGCCATGCCCGAA | EGFP | FAM labeled EGFP qPCR probe (TMF959-960) |
| (SEQ ID NO: 12) | | Zen/FB double quencher. |
| CACGTGGGCTCCAGCATT (SEQ | ApoB | For to TMF963 oIMR1544 genomic Probe |
| ID NO: 13) | | (TMF964) qPCR. |
| TCACCAGTCATTTCTGCCTTTG | ApoB | Rev to TMF962 oIMR3580 genomic Probe |
| (SEQ ID NO: 14) | | (TMF964) qPCR. |
| CCAATGGTCGGGCACTGCTCAA | ApoB | HEX labeled ApoB qPCR probe (TMF962-963) |
| (SEQ ID NO: 15) | | Zen/FB double quencher. |

### G0 Backcrossing

To isolate the transgenic allele, PCR-positive G0 founders were backcrossed onto B6SJLF1 animals. Oct4-GFP offspring were backcrossed up to G3 generation to stabilize the transgene copy number.

### Embryo Harvest for Imaging

Hemizygous (HEMI) males were crossed with B6J females, and HEMI females were crossed with B6J males or Tg(Pou5f1-EGFP)2Mnn/J (Jackson Laboratory, Cat #004654: TgOG2) HO males. B6J females and TgOG2 females were superovulated by subsequent hormone injections (PMSG: 3 days prior to mating, and 5 U of hCG: 1 day prior to the mating). Animals were housed together over night (for 1 cell embryo harvest) or two days (2 cell stage embryo harvest). Embryos were harvested and cultured in KSOM droplet overlain with equilibrated mineral oil, at 37 deg C., 5% CO₂, 5% O₂, 90% N₂ in a PLANER BT-37 incubator (Origio, Malov, Denmark).

### Imaging

A Nikon microscope was used. Magnification was set at 11.5×. Fluorescent imaging parameters were fixed at the same gain/exposure time to compare the signal intensity between the litters or each embryos. Bright field image was taken at the auto exposure setting.

### Sperm Cryopreservation

Sperm cryopreservation was performed with established protocol, as illustrated in Nakagata, N. (2011) Cryopreservation of Mouse Spermatozoa and In Vitro Fertilization. In: Hofker M., van Deursen J. (eds) Transgenic Mouse Methods and Protocols. Methods in Molecular Biology (Methods and Protocols), vol 693. Humana Press.

### Results

Micro-injection was performed using B6SJLF2 fertilized oocytes as donor strain. 142 embryos were injected, 36 pups were born, 7 G0 animals were confirmed to carry the transgene.

To isolate the transgenic allele, 7 positive G0 animals were each backcrossed with wild-type B6SJLF1 animals. Five of 7 founders transmitted the transgene array through the germline (subsequent lines or offspring from the five founders were named respectively as Line A, B, C, D, or E). Initially, genotyping was performed by conventional PCR to detect the mEGFP insertion in the mouse genome. After difference in mEGFP expression intensity was observed in each line, a qPCR-dCT assay was developed to measure the relative copy number of mEGFP in each line.

During breeding to develop the independent lines, an unusual fluctuation in the copy number of the transgene in Line B was observed between generations. Even at G2 generation, variation in the mEGFP copy number was observed within the same litter. The cause of the transgene copy number fluctuation remains unknown. However following backcross of the transgene in each line to B6SJL F1 wild-type mice for at least two generations, the fluctuation in copy number has disappeared. It is possible that the fluctuation occurred due to intra-chromosomal recombination involving the transgene array.

Relative mEGFP copy number was determined by normalizing the mEGFP signal to an internal control (diploid copy of ApoB gene).

To determine if HO mice were viable and fertile, and to try to increase the OCT4-mEGFP signal, G3 HEMI males were crossed with G3 HEMI females. The genotype was determined by qPCR-dCT method: HO genotype was determined by the double dosage of GFP transgene compare to the HEMI control of each line. HO animals were confirmed as viable for Line A, B, C, and E. A Chi-squared analysis shows the genotypes of offspring from HEMI intercross of line C follow expected Mendelian ratio. Results suggested that HO line A embryos had increased viability compared to HEMI and WT, and line B HO embryos had decreased viablility, HO of Line A and C were confirmed fertile. Although HO of line B could mate and produce embryos; however, Line B HO females had not produced any pups when crossed with HO or HEMI line B males.

HEMI or HO males were crossed with superovulated B6J females (paternal line). Embryos were harvested and mEGFP signal observed by conventional fluorescence stereomicroscopy, or confocal fluorescence microscopy. Embryos were harvested from 5 lines. GFP expression was examined under the Nikon stereo microscope. The embryos were cultured from 1 cell stage to up to blastocyst stage and observed daily. The expression was observed from 8-cell stage (96 hr hrs) up to blastocyst stage (120 hrs). This was similar to OG2 GFP expression. The expression level in each line was proportional to their mEGFP copy number. Line B had the highest GFP expression level, and Line D had the lowest. The mEGFP expression was observed in a punctate pattern in each cell. This pattern was distinctly different from OG2GFP. This was due to the IS construct has mEGFP fused to OCT4 rather than the mEGFP simply being produced from the Oct4 promoter as is the case in the OG2 line.

### Example 2-Development of a Quantitative Bioassay for Assisted Reproductive Technologies

Pou5f1-GFP transgenic mouse lines expressing GFP-tagged POU5F1 were generated to utilize nuclear localization of POU5F1 and to detect adverse culture conditions and epigenetic defect during preimplantation. Pou5f1-GFP expression were also used to visualize blastomere nuclei for cell counting in live cells. Pou5f1-GFP embryos were cultured for 96 hrs under optimal or suboptimal oil overlay to observe POU5F1-GFP expression at different stages of mouse embryo development (from 2PN to expanded/hatching blastocyst). (Experiments, n>3).

Pou5f1-GFP one-cell embryos (fresh or frozen) were cultured to blastocysts in test conditions uninterrupted up to 96 hours in Continuous Single Culture Medium-Complete (CSCM-C, FUJIFILM Irvine Scientific) with control or suboptimal oil overlay (5, 7.5, or 10% adulterated oil) and observed daily. B6 one-cell embryos typically used in the standard mouse embryo assay (MEA) were also cultured in parallel. These embryos were evaluated at 48 hours (%>8-cell) and 96 hours (% Blastocyst).

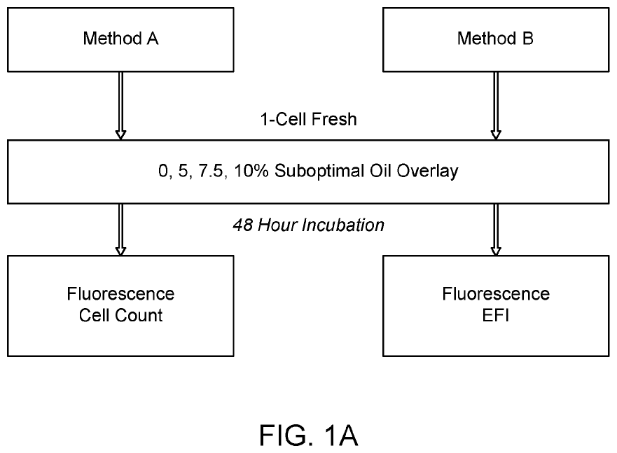

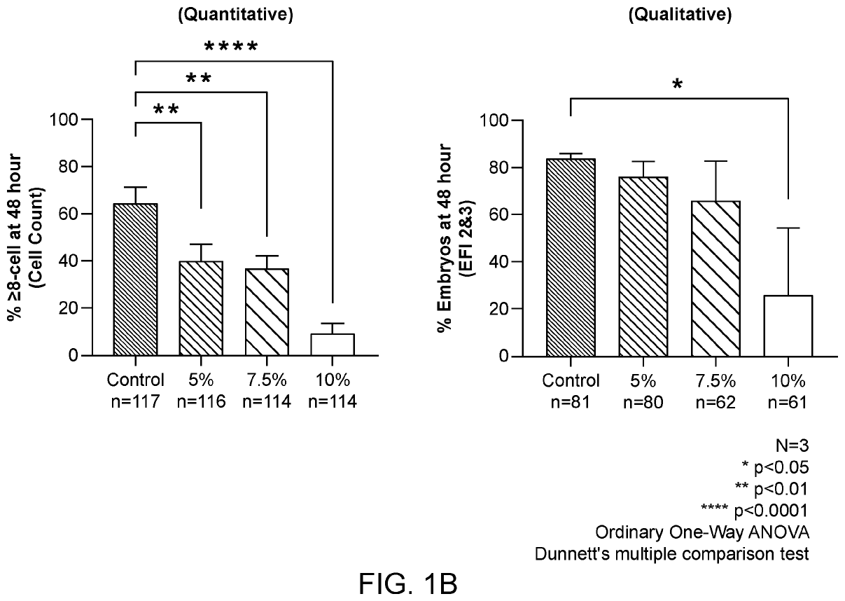

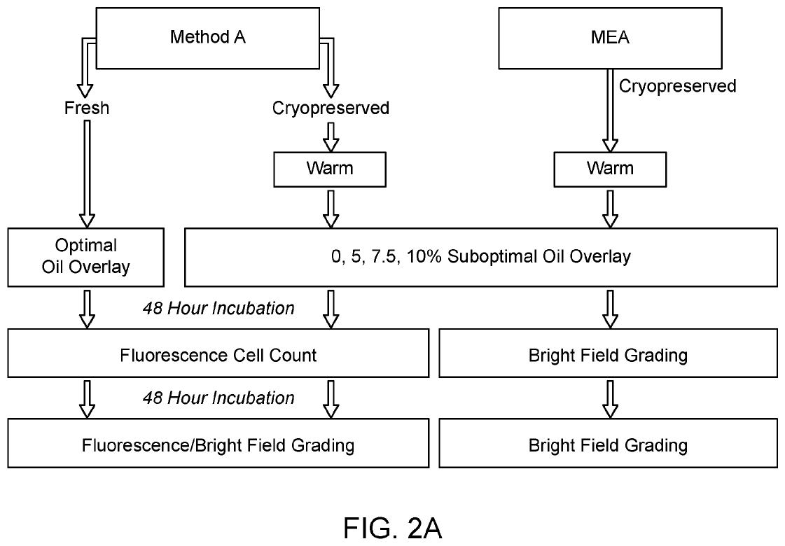

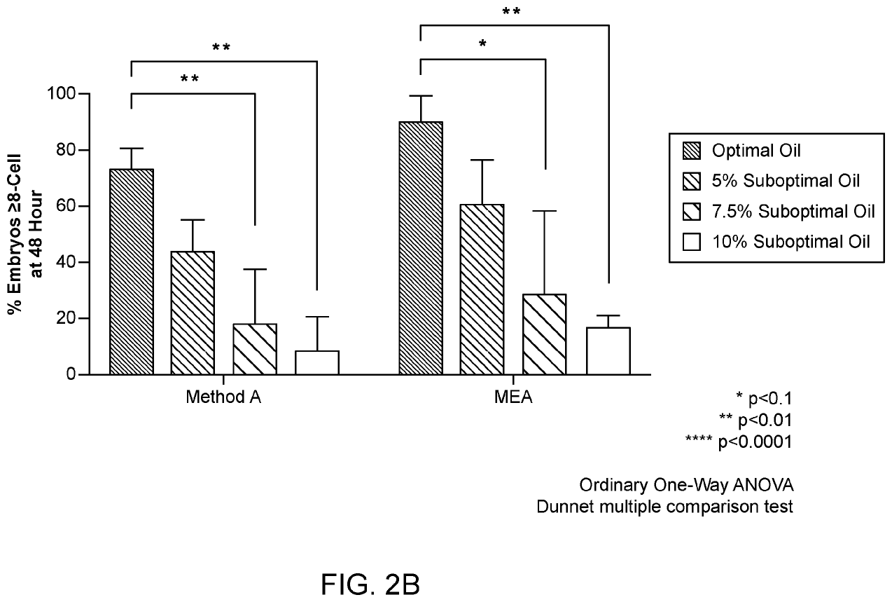

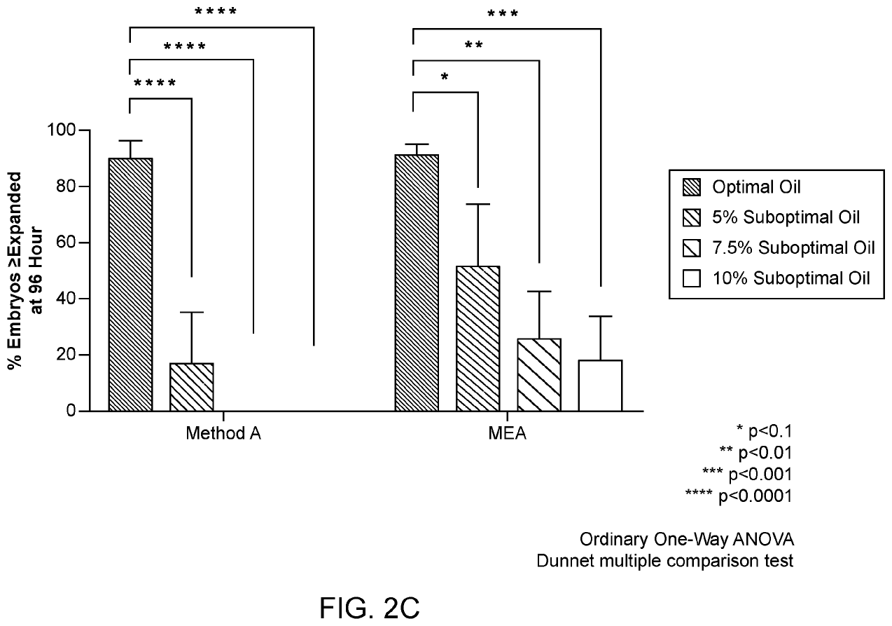

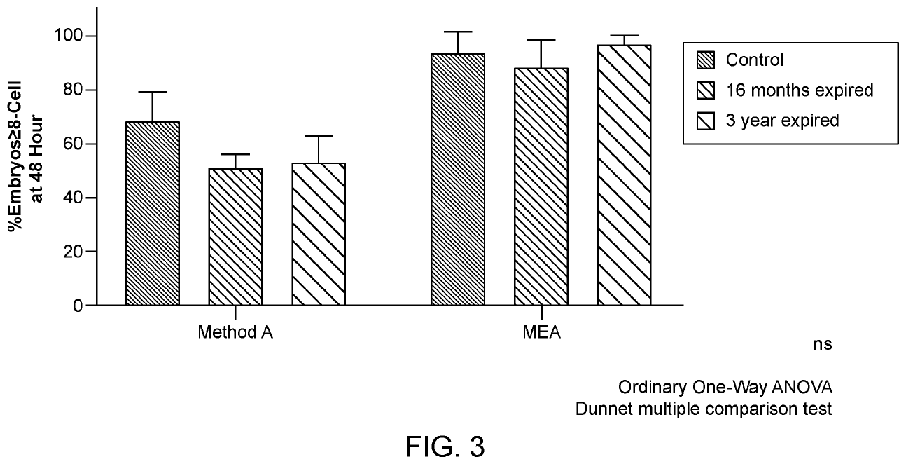

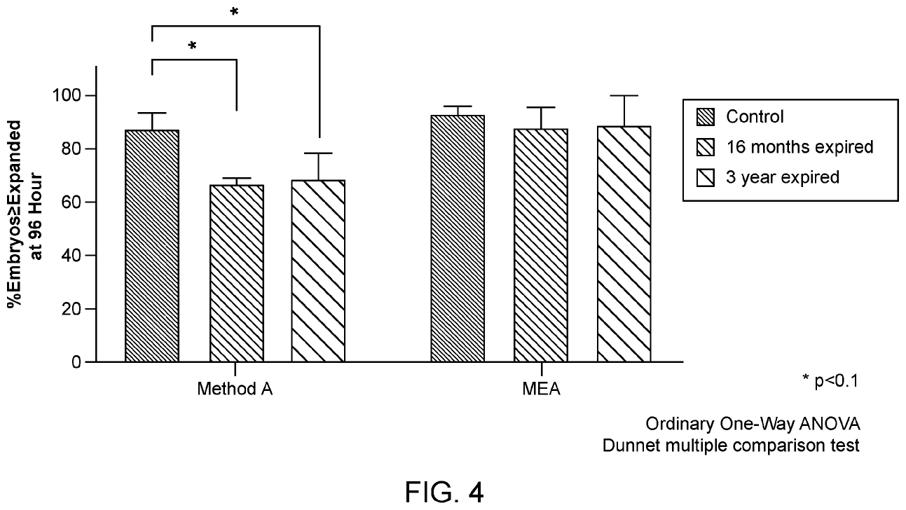

Transgenic mice expressing Pou5f1-GFP were viable and fertile, and successful germline transmission and temporally and spatially regulated gene expression were confirmed. Zygotic Pou5f1-GFP gene expression started around the 4-cell stage and peaked after culturing for 72 hrs. The nuclear localization of POU5F1-GFP in mouse embryos enabled to visualize nuclei of blastomeres and count cells in live cells as soon as GFP expression was detected around 4-cell stage. The Pou5f1-GFP embryos cultured with suboptimal oil overlay showed a noticeable delay in development (at 48 hrs and 96 hrs compare to the control oil group). Mosaic patterned expression of POU5F1-GFP was observed in some embryos cultured with suboptimal oil overlay. Pou5f1-GFP embryo culture detected 5, 7.5, and 10% suboptimal condition with statistical significance while the standard MEA (>80% passing criteria) passed 5% suboptimal condition at rate of 28.3%. See FIGS. 1A-1B and FIGS. 2A-2C. There were no difference in performance between fresh and frozen Pou5f1-GFP embryos. Pou5f1-GFP embryos cultured in adverse culture conditions had reduced subjectivity of embryo grading, leading to adverse epigenetic effects. See FIG. 3 and FIG. 4.

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. A transgenic mouse comprising stable expression of a fusion protein comprising an enhanced green fluorescent protein (eGFP) tagged octamer-binding transcription factor 4 (OCT4) under transcriptional control, wherein gene expression of said fusion protein is stably transmitted through germline DNA.

2.-3. (canceled)

4. The transgenic mouse of claim 1, wherein the the eGFP comprises an A206K mutation.

5.-6. (canceled)

7. The transgenic mouse of claim 1, wherein the eGFP is operably linked to the C-terminus of an OCT4 locus.

8. The transgenic mouse of claim 7, wherein the OCT4 locus comprises a deletion of a proximal enhancer element.

9. (canceled)

10. The transgenic mouse of claim 1, wherein the germline DNA is from a germline selected from a sperm, oocyte, stem cells, or zygote.

11. The transgenic mouse of claim 1, wherein the transgenic mouse is viable and fertile and the fusion protein gene expression is stably integrated in to a transgenic mouse zygote.

12. The transgenic mouse of claim 11, wherein gene expression of the fusion protein in the zygote starts from a 2-cell stage, 3-cell stage, or 4-cell stage cell development.

13. An embryo expressing an OCT4::EGFP fusion protein, wherein an oocyte is fertilized with a sperm comprising the OCT4::EGFP fusion protein, wherein the sperm is derived from the transgenic mouse of claim 1.

14.-15. (canceled)

16. A method of producing a transgenic mouse comprising, microinjection of a zygote with a bacterial artificial chromosome (BAC) construct, wherein the construct comprises a reporter gene that encodes an enhanced green fluorescent protein (eGFP) and is operably linked to a mouse OCT4 locus and the zygote is implanted into the reproductive tract of a surrogate mouse, thereby producing the transgenic mouse that stably expresses the reporter gene.

17. (canceled)

18. The method of claim 16 or 17, wherein the reporter gene locus is stably transmitted through germline DNA of the transgenic mouse, wherein the germline is selected from sperm, oocytes, stem cells, or zygotes.

19.-30. (canceled)

31. The method of claim 18, wherein the construct mediates expression of an OCT4::EGFP fusion protein that is stably integrated into the zygote.

32. A method for assessing a product used for assisted reproductive technologies (ART), treatment of a disease, drug screening, or immune modulation, comprising: (a) obtaining a transgenic embryo comprising stable expression of a fusion protein comprising OCT4 fused to an enhanced green fluorescent protein (eGFP); (b) culturing the transgenic embryo; (c) evaluating expression of the fusion protein; and (d) determining acceptability or failure of the product.

33.-35. (canceled)

36. The method of claim 32, wherein evaluating comprises visualizing nuclear localization or cytoplasm localization of the fusion protein.

37. (canceled)

38. The method of claim 36, wherein the evaluating further comprises determining a temporal and spatial expression of the fusion protein.

39. The method of claim 38, wherein the evaluating occurs at a 4-cell stage, 8-cell stage, or blastocyst stage, preferably at the 8 cell stage.

40. The method of claim 39, wherein the fusion protein is predominately localized or expressed in the nucleus at the 4-cell stage, 8-cell stage, or the inner cell mass (ICM) at the blastocyst stage.

41.-48. (canceled)

47. The method of claim 40, wherein the product is not acceptable if there is less than 40%, 30%, 20%, 10%, 5%, or 1% of nuclear localization or expression of the fusion protein at the 4-cell or 8-cell stage or less than 40%, 30%, 20%, 10%, 5%, or 1% of localization or expression of the fusion protein in the ICM.

48.-54. (canceled)

55. The method of claim 47, wherein the product is a protein or a gene associated with a disease or development of an embryo.

56.-57. (canceled)

58. The method of claim 32, further comprising (e) obtaining one or more embryonic stem cells from the transgenic embryo and culturing the one or more embryonic stem cells to generate a plurality of embryonic stem cells; and (f) incubating the plurality of embryonic stem cells with a drug, evaluating the expression of the fusion protein, and determining acceptability or failure of the drug.

59. (canceled)

60. The method of claim 58, wherein the drug is for use in the treatment of a disease or modulating an immune response.

61.-62. (canceled)


SEQUENCE LISTING (16 sequences)

SEQ ID NO: 1 - protein, 351 aa, Mus musculus
AGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSN

SEQ ID NO: 2 - protein, 239 aa, Unknown
  Description of Unknown GFP protein sequence
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 3 - protein, 16 aa, Artificial Sequence
  Description of Artificial Sequence Synthetic peptide
SGGGGSGGGGSGGGGS

SEQ ID NO: 4 - protein, 606 aa, Artificial Sequence
  Description of Artificial Sequence Synthetic polypeptide
MAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQRKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSNSGGGGSGGGGSGGGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 5 - DNA, 19278 bp, Artificial Sequence
  Description of Artificial Sequence Synthetic polynucleotide
gggcctactaagtgttcactatgcagcaggggctagcctgaactcccagatagcattactgttatatataaataatagcattactgttattttaaatttagtttgttatgtgtatgagggttttgctttcctgtgagcatgccctcaaaggtcagatgagggcattggatcctctggaactgaagttccaaatgattgtgagccatccatcattcaggtgctgagaactgagctcaggtcttctccaagagcagcaggtgctatttaaattctctctcgccgggcgtggtggcgcacgtctttaatcccagcactctggaggcagaggcaggaggatttctgagttcgaggccagtcctctccctcttcttcttcttcttttttttttccgagacagggtttctctgtgtagccctggctgtcctggaactcactctgtagaccaggctggcctcgaactcagaaatttgcctgcctctgcctcccaagtgctgggattataaattcttttttttttttaagatttatttatttattatatataagtacactgtagttgtctccagacgcaccagaagagggcgtcagatctcattacgggtggttgtgagccaccatgtggttgctgggatttgaacttcggacctttggaagagcagtcgggtgctcttacccactgagccatctcaccagcccctggattataaattcttatttgtacagtttttgttgttggttttgtttgtttgtttgtttcaacacttgggattgaatccagggtcattggagtgtttggcctgggttctgctgctgagccacactccatatacttgcatggattgtgtcttttattctatgtggtggtggtggtggtggtggtggtggtggtggtggtggtggtggtggtggtagagggaactcacccagcccacctcctctggtttcactcctccacacacacccttcatccctgtccttaccccctgccagtaagaaactccacaggatgtctctctaggagtctgagaaaaaagcactaagagaacaagctgtttgttttttggggttttttttgggggggtgggggggggagttagaactcaggaaatcagaacagggctaggtgtggtaatgaatgacatagatctagtcactcaaaggctgtgtacctctgaagttgagactagcctggtttatataaaaagtaacctggtctatataaaaaattctaagttacgccgggcgatgatggcacatgcctttgatcctagcacttgggaggcagaggcaggcggatttcgaagtttgaggccagcctggtctacaaagtgagttccaggacagccagggctacacagagaaaccctgccttgaaaacaaacaaacaaacaaacaaaaaaccaaaaacaaccaaaacaaacaaacaaaaaaaccccaaaaccaaaacaaaacaaaacaaaaagttctaagctatgtagaactcttggggcactcgtgatcaaagtctaagcattgaaaatctctaaatgccaggcttggtggcgcacgcacttaataccagcacttgggaggcagaggcaggcagatttctgagttcgaggccaacctggtctacaaagtgagttccatgacagccagggctacacagagaaaccctgtctcgaaagacaaaaaaaaaaaaaaaaaaaaaaaaaaaaaatctctaaacaagccctatcaatcttcccttgggggtatggactgttatctcttcatcataagaggaaagcgaataggtggtgtgaaggaggtctttggtggtgcaggagggagccactggctgcaggggacacagggtacagcatggctccttagttggacatctgcttgacagaatgagtgtctatctatctgtggacttgggaatcaaccccaggtattccaggaggtaagaagaaccactctctgaaccagggtggtcttttgagccaccagaaaaaacatccaccatgtaattgtaaccaagtaaccagtcataaccccctgcaacagcgcccaggagacttttgaatctgaaacaagcagaaccacagagagggggaggaaaggttatcctgacccagagaggcaaatcctctctggaaccccgggagctaggtcagatgctaagggcatttgtggggagggctgggagagaagggccctaggctagcagcaggtcctccttccctttaacctctggccatgttgggtagggactctccttgacaaacctgataaccaggagttcatttccgagcacccacacagtggaagaagctagcccatcgcctaaagttgcccttttgttctctttttctctctctcacacaaatttttaaattttattatttatttattgacataatttctctatgtatcccaggctgtctcagaactcactttgtagaccaggctggcctcaaacttagagatgctcctacctctgcctcctgagtactaggattaaggatatccatcacaacacctaactgtaataaaaatttcaatttttttttctctcttagcctcagctgcaatcacctcttttagccagtatacctgcaggagctccagtccataggcgggctctcctaatgcctgaatttgactctcaaagtgcattcacttcctctcattacagctcttctgcagactcttttatatcaggaatctcaaaacttgggttgggcatggtactcgcctttaatcccaggaggtggaggccagcctggtgtacaaagtgagttctaggtcaggcaggactgtaatgtctactccttacatgaacaggatagggcctggcggggagagaggggggaagctaggtgggagtaagggtggaaacgggcaatcgtgtcaatggaagaagtaaatgatcaagacaataggtctgttcacagcattagaaccacccaaatggtcatttggcatcggagtcctctccctcctcccaatccccccccccatttgcttatctatttatttttattttatgtatgcaagcatttttcttttttctttttctttctgggtttttcgagacagggtttctctgtgtagccctggctgtcctggaactcactctgtagaccaggctagcttcgaactcagaaatctgcctgcctctgcctcccaagtgctggaatcaaaggcaagggccaccacacctggcgcaagcatttttcaaagatgtatttgtttatatatgtgagtacattatctctctcttcaggcacaccaggagagagagagagagagagagagagagagagagagagagagagagagagagagagagaatcagatcctgtcacagatggttgtgagtcaccaagtgggagctgggatttgaactcaggacctctggaagagctgtcagtgcacttaacctgtgagccatctctccagtccttttcctctctgttttttttttttttttttttttttttttgacacagtttaaccatgtagcttaggctaatcttcagtttgctgtgcctcagcttctgagtgtccacttgggattgcaggagtttgccgccttgctcagcttacagcttgggatgctcttctcacagacatccacgtactagtactgaaagacaaagacaaacaccggtttgcttgcaaactgctcaagtcttgtctagtttgattaggcttgagaggagaccttgccagctttcccaagaagactgtgtggaggttggcggtagaaggtggggtacagaagacttgctttcatctcacaaggtgccccacccatctctctaacgcccaatttttctttacagaggcacagggacaactccctaccccgacccatgtggagtgagaagggcaggaggatgcatgggaatagggctgtagttttcttttcaagttggaagaaaatggagagttaggccagggaaggtagtgacacccccctggggtgtcacacacacacacacacacacacacacacacacacacacatacacacacagggaattctagcactggtggtgtgagcaagtaggtagctatatgtagtaatataacaagatttattttgctgagagtcatcacataaggttcacactctaagccagccaggtggtggtggtggcacatgcctttaatcccagcacttcggagacagagacaggaggatttctgagttcgaggccagcctggtctacaaagtgagttccaggatagccagggctacacagagaaaccctgtctcgagagaaaaaaaaaaaaaaaaggttcacactctgtaaattcctaactaatgtctcattcctgtctccccacagctgctcacctatgtgacatattttagcagaaggtcaggtccactctcttaatctcttaagagtgtctgtgatttgagggacaggatcctagcggggagactctccacctacaaggcagatcaaggtggctggttatcttgaatgagtgatgtcgtggggttctggttatcaggggcagccttgggtgtagtggtgaagccatttctgcaagtctaaaaggcatttggacactgctcatcagaactgggaaggcaagaggatcagaagttcaagggccttcttggctacgtagagtttaggggcagcctctgctacatgtaaatttgtctcagaagaaaaaaggaaacgggcagtagtggcgaatgcctataatcccagcacttgggaagcagagggcaggctgatttctgagctcaaggccagcctggtctacagagtgagttccaggacagccagggctacacagagaaaccctgtctcagaaagaaaaaaaaaaaaagggagaaatagaactcatttatttcaaagggagcaggcacattccgcaagccttctgttctgctggtgacctattcctaccttcagcttctagatctttgttttttcttttaatttttattgtattgtgtgtgtgtgtgtatctgagtgcaatatcaatatcgaacctctggagatcaaatgcttctttgaaatggaccccagggatcaaatccatggtcgttaggctcagcaagtgtggttacttgctgagctgtcctcaggttcctctcaggcctcccctcaagtctccccctccaaccccccccccctcctgccccctgcaagtctttttgattaggcaagaaaaccaagacaaggaaagggagatgcagttagctaaggaatctatgccagccagagaaactacctcctcccttccagaacatctggatttgggaagagacgttgctggtcccagggcggctgggggttggggttgggggagggggatgctaaccagcaaggaagctgttcctggctggggcaggcctgactgagctcatgtcgctgaaactcctcatttctccctatggcttcatagggagacccagcctggatgctaacacgagtgatttccctgctctagtctagtgtcctccgtgagtccatttaactgatcacccagtctgtgaggaggtggctgaactcacagtaagaaagctgtgggggtcaacgcctattgtttgtttgttttgttttagacaaggtctcctgctgaggctggctcaagctggcctggaggactcttgtgtttaaggctggccttaaattctctttaaaagaaaatcatgtgtatatatgtgtgtccatgtaactgcagatgaccacagcagccaagagatttctgttctcctagctgtaaaccacccaatatgggtgctaggaacagaattttaaaagggtcctctgaaagagcaatgtacactcaaatgctgagttctttcccagcccctagccttggacctttgttcttatcacttccaacgcccaagggcaggcattataggtgtggcattccgcatctggcttcccaggataccttttcatgctggtggaccatctctggctggggacgtgtgggcttctctgctgtctttggttctccagacagaactccgagacagatcttgacttggttctaaaatacaggtggtttgtggcaagttaacgaattttagctcaaatttggggtatttaagataccatggtgactcttttttgtttgtttttctttctttcttttttttttttaaaaagatttatttattattgtacgtaagtacttcagacaccagaagagggtgacagattttttttttttttcgagacaggatttctctgtgaagccctggctgtcctggaactcactctgtagaccaggctggcctggaactcagaaatccacctgcctctgcctcccaagtgctgggattaaagccatgagttgggaccgcacccggcccaaagtgactcttaaagggggcagagtggcacatattttcaatcctagcactcaggaagcaaaggctggtggatctctgtgagttcaaggccagcctggtctacagagtgagttccaagccatctaaggctatgtagggaacccttgaatcaaacccaaaagttagtctgatgattttctaagacccaggaggcaagaaactggatcagatgagccaacaggtctgctgtcccatctccagggccaccaggctcacagctcgggaccaggctagggcacatctgtttcaagctagttctaagaagacttgggacttcagacaaagttgctgttaaggactgtattatactctaggcacgcttagggctaacctggttgcaaagccagtcactaggcagttaaaggactcagaatatgtctcttgtcctggccagtgagtcaccaaaagagaaatcacaatccataagacaaggttggtattgaatacagacaggactgctgggctgcaggcatacttgaactgtggtggagagtgctgtctaggccttagaggctggccctgggaggaactgggtgtggggaggttgtagcccgaccctgcccctccccccagggaggttgagagttctgggcagacggcagatgcataacaaaggtgcatgatagctctgccctgggggcagagaagatggttggggaggggtccctctcgtcctagcccttccttaatctgctattgaggaagctttgtgaacttggcggcttccaagtcgctgcctttatttaggtcttccaactaacctatggcactgttccacaatgaatgtatagaaattgggaggtgagcatgacagagtggaggaaacggaagattcatggagagggccagagagatggcccctcagccaccctgggggatgacttggacccatgtggtagaaggaggggacttccacacatgtgctatgtgtagctgtgtgtaggtacatacacacccttaaaataaaacgcaatttttttttcaaagtctcagggtgaatttggtgaagtcgatgaagctgaggcaggagaattatcaggagttcaagggcagcttgttttatagagaaaggttccatctctacctgatgaagactaccatcaagagacacccccgccccccagggcacctagagccactgaccctagccaacagctcaggcgggctgggcccaggctcagaactctgtcctggctatgtacactgtggggtgctctgggctttttgaggctgtgtgattcaccctggggccttcgttcagagcatggtgtaggagcagacagacaaacaccatcccttgcagacaggcactctgagggctattctcttgcaaagataactaagcaccaggccagtaatgggatcgtgacccaaggcaggggtgagaggaccttgaaggttgaaaatgaaggcctcctggggtcccgtcctaagggttgtcctgtccagacgtccccaacctccgtctggaagacacaggcagatagcgctcgcctcagtttctcccacccccacagctctgctcctccacccacccagggggcggggccagaggtcaaggctagagggtgggattggggagggagaggtgaaaccgtccctaggtgagccgtctttccaccaggcccccggctcggggtgcccaccttccccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggaggtaagtgaagggacttggctgggctggcagaggcagcagtgaagggaattgggaacatgtagggtagccaccctgcctgccaaaggtggtgatggctgccgggcctcctgagaagcacgacgcagtgtggactagaacccagaattgcaagaatcagaaaccggcctggattgtttcggcctggcccttgtcatgtaggtcacctaggcctggcctgtgtcccgacacttgcttcatgccatcactgtctgtacaccagtgatgcgtgaaaatcagcccccccccaaaaaaaaaaacatatcagcccctctggggacttggatcacagtcggacccaggaacttggccttaaggttaggcatggctgggggggtaaaaaatggtgcttatcctggagttattgttactgaagaggttgggtgtgactggctgctgataggagctcttgtttgggccatgtgtggagtagggctcaccttcagtcaagtttacggcctgtctactttagcctcagactccatgagtcacctttacacgagcagacccttgtagtgcctgaggtgcagatctgatcgatttcagcctttctacctttccttgtaaacaagaaagggacacccttgggtaggggagttttatctccaggccatcttaagatcattctgtgagtgcacgggccttgcttagtgtctgatggcctacagccagcactctggagcaagtgtaagcaattagccttaagaacaaggtgcgagtggataccgatgcccgccgggagttccgacagcttagcgattgttgtagcaggagtcccctccctaagtgccagtttctgtgttatctcaggtcctgtatgccgccgggagtcccctaggaaggcattaatagtttatctcacatcttaaatggcccttaatgaagcaagagatttgaaccttagttaagctaatcccaaatcctcaaaataggatttagaaaagccaaagacactgctgagggcgattacaagttttggtcttttgaggagcagttggagatgaaagtctgtctgaagccgagagaatccttttccattgaaatggcattgaggtgtgcctcactggctgctgcttctgtctgtgccctgggttggccagcctttgtggagcacctcagccctccatcctggacctttgctccaacaacctgctcctcttccgccctcaaggctgacttgcatctccccagatgactgcctccatttctgtcttctgttagagacagaaaagcctgagaaaccgacagccattttggggggggggggtccggttcacacgctgcaacttagaaagcacactcaactggccatctgttataccctccccacctggtcccaaccatcactgtgtactactgagaagaaggcagccttagccacaccctcgagtgcccctgccgttctattgctcatacatcgattgatatccctgtttcaactttgaaaaaaaaaaatttttttttttttgtggtgtgtgcatgcctgctactgtacacctgtgggcgtcagaggtggtcctctgcaccctccggccagtaccgcatccagggtgagtcagatgatttcctgtggtttgggcctcaaggcttctcacctccagaggcttctagcctgctgccttgctttctctgtcgcactctagtacagcaggagttttcttcgcactccggagtgttgtcagctcctggggcatggacatttggctacttagagtgtgctgtgtaggttttcatttagagctgaacagagggatggatcttattaccccagcccttgagacactgaggcaggagagcttcctagtgagtccctgtttcaatatcttcactaatactgtgtcatactttgggactttctttcttcctttctttcttttgattttttttttttttatatgagtacagtgtacctgtcttcagacacacaccagaagagggcatcagatcctactacagaaggttgtgagccaccttgtggttgctgggaattgaactccggatctccggaagagaagtccgtataccaacttctgtattagtcagggttctctagagtcacagaacttatggacagtctctagatagtaaaggaatttattgatgacttacagtcggcagcccaattcccaacaatggttcagtcgcagctgtgaatggaagtccaaggatctagcagttacttagtctcacgcagcaagcaggcgaaggagcaagagctagagcttaactgctgagccatgtgtttcttgagtaaagggattacatgctcgttcgtctggtcaattctgcagccttaaaacttcttcagaatagggtgacattttgtcctcagtggggcggttttgagtaatctgtgagcagataggaacttgctggggtactgcacagaactctgggtagtgtggtactgtagatggctaggttctggggggggaaagagccatctatgtcacctaggaatagagtgaataacatttatataatcagaccagcccttgaggaggctgagatcttttcatggggcaccctagggtcacagtcccagctggtgtgactctgacaagtctgcctttctcactacagtcccaggacatgaaagccctgcagaaggagctagaacagtttgccaagctgctgaagcagaagaggatcaccttggggtacacccaggccgacgtggggctcaccctgggcgttctctttggtgggtctcccccagcatgttctgatctcacggctcttaatgtaggcgcaagggggtggggcatcttaggagctgcttctccacaggtaagggaggattagacgcttgtagcttgaactgtcagaggtgggggcttgggctcccttcttgctgcctcactcactctgtttgatcggcctttcaggaaaggtgttcagccagaccaccatctgtcgcttcgaggccttgcagctcagccttaagaacatgtgtaagctgcggcccctgctggagaagtgggtggaggaagccgacaacaatgagaaccttcaggaggtgaggagtggcaggatgtgtgcaatgtctgccaggcacagtcccttctgctgcttccattcctggcttgaaactcctccctctccaaccggagctcgcaggagaagttctgtgtccttattctgctgctatgaattggaatccagagccttaacatttgctaatcaatcaggctctctccttctgagtcaccctctgcccccaccagcctgacaatggtccctccccagaaccccgtctagtgctggtgaaggctcagacctaggtctaccagccccttccagagcccctttcagtaacccctggctctggggccacatccagtcaatgctcccttagcacaatcccttagcggtttgttcttcagtcccatctcaaggtggggctgttgccaagtcaaatactaaagttgctcttgtcgcccccatcttcccctgcccagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtgagtgccaagatcctgccctgtggtacctggatgtttccctgttcccattccccaccccccccacccccccacccccaccgccgccaccgctgactgcagcatcccagagcttatgatctgatgtccatctctgtgcccatcctaggtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaactctggcggaggcgggagcggggggggtggctccggcggcggagggagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagcttagcaaagaccccaacgagaagcgcgatcacatggtcctgctggagttcgtgaccgccgccgggatcactctcggcatggacgagctgtacaagtaatgaggcaccagccctccctggggatgctgtgagccaaggcaagggaggtagacaagagaacctggagctttggggttaaattcttttactgaggagggattaaaagcacaacaggggtggggggtgggatggggaaagaagctcagtgatgctgttgatcaggagcctggcctgtctgtcactcatcattttgttcttaaataaagactgggacacacagtagatagctgaattttgttttccttcagttcctagagagcctgcggttggagaaagccagtaatggattctcaaaccccaggtgatcttcaaaacaggcgccattgaaaccattggagttccacaaaatgcccagggatagttggggttggagcccaacctatagaggaaggcattgcatattcgccatcctagaggcggtaagtctctgctagctgatggacatcacctcatagccattgtctggcagccgccttctttcctcttgtcactctgggagttctggtgggcttatactttaaaaaaaagagtttttttgggggggttaagatttattttatttatatgggtacactgtagctgtcttctagacacaccagaagagggcatgggatcccattacagatggttgtgagccaccatgtggttgctgggaattgaactcaggacctctggaagagcagtcagtgctcttaaccgctgagccatctctccagccctcaaactcttttttttcttttccttcaagatgagttctgtgtagtcctggcggaccaggttggcctcagatcagcctgcctctgcctccgcagtgctgagattaaaggcccgtgccactctaggctaaattgttatgcttctattctagctgatgaccaccttttttgggcgtagtagtgctgggagtagggtctgtacacatgtctacaatgccagaataggtcaaaggctttagatctcaaggaactggatttatagagagttgggagcagccatgtaggttctgagaaccaaacctgggtcctctgcaagaagagccattggctttttgtttttgtttgttttgagacatttctcggtgtagccctggctatctggaactctgtaggccaggctgtccctgaactcagatccagtctatccatccctgccttccaagagctgggattaaggtcatgtaccaccacaggccagctagccatagctcctaactgctgaaccatttatttatttatttattttatttttttggtttttcgagacagggtttctctgtatagctctggatgtcctggaactcactttgtaaaccagtctggcctcgaactcagaaatctgcttgcctctgcctccccagtgagtgctgggatcaaatgcgtgcgccaccactcccagcttaagtctttattttttaaaatgttatttattttggggctggtgagatggctcagtggttaagagcactgactgctctgccagaggtcccgagttcaaatcccagcaactacatggtggctcacaaccatctgtaatgagatctgacgccctcttctggtatgtctgaagacaactacagtgtacttacatatatatataataaataaattaaaaaaaaaaaagagaggaggagccaagcagctcctttagaaaaaaaaaaagttatttattttatgtatttgagctgtcttcagacacaccagaagagggcaatggatcccattacagatggttgtgagccaccatggttgatgggaattgaactctggacctctggcagagcagtcagtgctcttaaccgctgagccatctctccagcccccaaagccaagtcttaaagcatttttgctgctgaatgtcagccctaccagatctctgcctccctcccctcccctccccccagtatctcatgaagaccaagctggcctggacacagtaagtatgtgcatatttatgtgtgaagattgtcacaatgtgaggaaaaaagttggttccctccatggtgtgggtcctgtgggtcaagtccaggtcgctaggcttggcagcaagtgccttgactcatagtcttctcctgcccaactccatgcttggtttccacgagcccctgtgctatggagaattccatctccaggcctcaccaaatctaatctccccatcctttgaaaagcagacttagattcaacgcaagtggatgaaacagtttattctttatttgggaataaagactaagctctgaaaagctagtcccagagactcagctggtggtgatactagctagcggtggcatgaggatgccttgggaatgtgctctgggtccttcagggtgctttagccgatgccattcaagaacatgagtagggttagggtattgtggcagagcacttgcctggtatatgctggcttcagcaaaataaaaccataccttctaggaatggtttctgggaccggtgctctaactgcaggtatcctggcatccatggaggcaaggctttattccttgtgactgggcttgtagctcactggaaacttggaggctgcaacatctttggcaggaaaccatcttttctgtcacttcatttgcaagcattctccagccttgagtcagtctttagcaatggacctttccctgtggtcattccctttggagaaagacattcctcaaagtccatggtaactttgaatgagtgttttgcatgtacacatgcgtgagtgtgcatgcgctctcacacacgcacgcacatgcacacgcgtgcacacacacacacacacacacacacacacacacacacacacacactgcttcagcccttaggagccattcttctattattatgtttgagtgctctgcctgaatgtgcacctgcaggccagaagagggcatcagatcccttttagagatggtcacaagccatcatgttgttgctggaaattgggacttctggaagagcagccagtgtacccttaattgctgagccatcttactgcccaagatacattcttacactgtgcctgaccctgagccacatctgtgtcctgactgcaaagtcaagatgccattatggcatcctggatgctatagccagtgcaggccagagggtaccagatgtcacagccatcacaccaacccaggctctgctctctagcaaaaagaagctggacaggactccctaagggagtgagtgttcctgagaaaccctttgagtaacttgcctctgggtaactggtagccagaacaggaggctaagactgggatataggaacttggagattagggatgttaagtagagcatacgcatagcacaaaagatacttggctttggatatgagctgttgacgccttcaatccatcacaactcccattctgaatgctctatcccgactacatgagggatttgaggctagcctggattacacagtgagaccttgtattaaaaaagagttgggtgtctcctccagagaggatctgggtttgaacctcagcacctacatagtggctagcaattatccctccagttcccagagaacccagtgccctcttctggcttctgccggtattgcatgcaagtgtgatacccaatcatgcaggcaaacaaagcagccttgaattgacctgctctcctctagttttgagacagtgttagtatggttttttatgtatagtgctgggactccaaacatgggcaagtcaggtgcttgctaggcagtgctcttctagtgagacatctctttgttccctgtctcccagattgctttgtatagtctagtcctaaccattgttcccacatagtagcttgtcatgcatttatgggtgaaagctaacctgggtgtctgctgtgcccgtgcaccccccttccctgccttctaagacctcagtctgaggctgttcaaagatctagaattcaaggtgctgacaggtgaccccacttacccactggctatcagagcagctctggcgaaaatgagacgttggcgatcgcgtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaatttgatatcgagctcgcttggactcctgttgatagatccagtaatgacctcagaactccatctggatttgttcagaacgctcggttgccgccgggcgttttttattggtgagaatccaagcactagtaacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatccctaggtataaacgcagaaaggcccacccgaaggtgagccagtgtgactctagtagagagcgttcaccgacaaacaacagataaaacgaaaggcccagtctttcgactgagcctttcgttttatttgatgcgatcgcctggagatccttactcga

SEQ ID NO: 6 - DNA, 24 bp, Artificial Sequence
  Description of Artificial Sequence Synthetic primer
atcttcttcaaggacgacggcaac

SEQ ID NO: 7 - DNA, 24 bp, Artificial Sequence
  Description of Artificial Sequence Synthetic primer
tcctcgatgttgtggcggatcttg

SEQ ID NO: 8 - DNA, 24 bp, Artificial Sequence
  Description of Artificial Sequence Synthetic primer
gagcttctggtattagcgttaggt

SEQ ID NO: 9 - DNA, 24 bp, Artificial Sequence
  Description of Artificial Sequence Synthetic primer
tccacaatgacaaagacatgaggt

SEQ ID NO: 10 - DNA, 21 bp, Artificial Sequence
  Description of Artificial Sequence Synthetic probe
ccacatgaagcagcacgactt

SEQ ID NO: 11 - DNA, 18 bp, Artificial Sequence
  Description of Artificial Sequence Synthetic probe
ggtgcgctcctggacgta

SEQ ID NO: 12 - DNA, 21 bp, Artificial Sequence
  Description of Artificial Sequence Synthetic probe
ttcaagtccgccatgcccgaa

SEQ ID NO: 13 - DNA, 18 bp, Artificial Sequence
  Description of Artificial Sequence Synthetic probe
cacgtgggctccagcatt

SEQ ID NO: 14 - DNA, 22 bp, Artificial Sequence
  Description of Artificial Sequence Synthetic probe
tcaccagtcatttctgcctttg

SEQ ID NO: 15 - DNA, 22 bp, Artificial Sequence
  Description of Artificial Sequence Synthetic probe
ccaatggtcgggcactgctcaa

SEQ ID NO: 16 - protein, 50 aa, Artificial Sequence
  Description of Artificial Sequence Synthetic polypeptide
  SITE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS